SEQUENCE LISTING (2 sequences)

SEQ ID NO: 1 - protein, 26 aa, Artificial Sequence
  Synthetic Sequence
GDYSHCSPLRYYPWWKCTYPDPEGGG

SEQ ID NO: 2 - protein, 12 aa, Artificial Sequence
  Synthetic Sequence
CSPLRYYPWWKC

(19) United States
(12) Patent Application Publication
(10) Pub. No.: US 2022/0001039 A1
(43) Pub. Date: Jan. 6, 2022

(54) COMPOSITIONS AND METHODS FOR DETECTING ACE2 EXPRESSION PROFILES

(71) Applicants: Daniel Thorek, St. Louis, MO (US); Hanwen Zhang, St. Louis, MO (US); Diane Abou, St. Louis, MO (US)

(72) Inventors: Daniel Thorek, St. Louis, MO (US); Hanwen Zhang, St. Louis, MO (US); Diane Abou, St. Louis, MO (US)

(73) Assignee: Washington University, St. Louis, MO (US)

(21) Appl. No.: 17/359,490

(22) Filed: Jun. 25, 2021

Related U.S. Application Data
(60) Provisional application No. 63/044,142, filed on Jun. 25, 2020.

(51) Int. Cl. A61K 51/08; A61K 51/04

(52) U.S. Cl. CPC A61K 51/08; A61K 51/0482

(57) ABSTRACT

Among the various aspects of the present disclosure is the provision of compositions of imaging agents and methods for use in imaging ACE2 expression profiles. The ACE2 expression profiles may be used for detecting, monitoring, and evaluating ACE2 associated diseases, disorders, and conditions.

### CROSS-REFERENCE TO RELATED APPLICATIONS

This application claims priority from U.S. Provisional Application Ser. No. 63/044,142 filed on Jun. 25, 2020, which is incorporated herein by reference in its entirety.

### STATEMENT REGARDING FEDERALLY SPONSORED RESEARCH OR DEVELOPMENT

This invention was made with government support under grant numbers CA229893, CA201035, CA240711, awarded by the National Institutes of Health. The government has certain rights in the invention.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE DISCLOSURE

The present disclosure generally relates to imaging agent compositions, methods of making imaging agent compositions, and methods of detecting imaging agent compositions.

### BACKGROUND

Originating from an outbreak in Wuhan, China in 2019, the severe acute respiratory syndrome coronavirus 2 (SARS-CoV-2) is the causative agent for the coronavirus disease 2019 (COVID-19). Since the first reported cases, global dissemination of the virus has resulted in the infection of tens of millions of patients and has caused hundreds of thousands of deaths. The illness is characterized most commonly by fever, cough, and fatigue; but can include headache, diarrhea, pneumonia, lymphopenia, dyspnoea, and hemoptysis. While SARS-CoV-2 resembles previous betacoronaviruses that transmit readily through humans, significant differences in the organ systems impacted (including the gastrointestinal tract, heart, blood vessel endothelium, and lower airway) greatly affect the spread and severity of the disease.

Although research into features of the virus has been undertaken with outstanding speed, many urgent questions remain unanswered, including factors that impact patient infectivity and outcomes. Addressing these issues is dire for men and women with existing illnesses, especially for those with cancer. Early analyses reveal that patients with cancer are at substantially greater risk of severe events including death, intensive care unit admission, and development of critical symptoms. The largest trial of cancer patients with symptomatic COVID-19 reports a staggering 28% mortality rate. Cancer patient age, cancer type, disease stage, and type of anticancer therapy have been implicated in less favorable COVID-19 outcomes.

Patients with cancer are at greater risk from severe outcomes from SARS-CoV-2. As illustrated in FIG. 1A, cancer patients exhibit more critical events from COVID-19. FIG. 1B summarizes the incidence of critical outcomes, including death, ICU admission, cardiac arrest, or usage of invasive ventilation with time following diagnosis of COVID-19. FIG. 10 shows that patients receiving different anticancer treatments have significantly different outcomes. FIG. 1D summarizes the incidence of critical outcomes with time following diagnosis of COVID-19 for non-cancer patients and patients receiving different anticancer treatments. These data demonstrate that worse outcomes for those receiving immunotherapy, chemotherapy, and surprisingly, having had prior surgery.

The projected new cancer diagnoses in the United States for 2020 are 1.8M. This immediately makes it a priority to develop and deploy novel techniques to address why cancer patients are vulnerable to COVID-19, and how their current anticancer treatments may affect viral outcomes.

The entry of SARS-CoV-2 into cells is mediated by an interaction between the viral Spike protein and the angiotensin-converting enzyme 2 (ACE2) receptor, as illustrated schematically in FIG. 3A. ACE2 receptor density in each tissue is a determinant of the severity of the disease in that tissue, invoking damage and failure of critical organ systems. Current approaches to assess ACE2 by ELISA or RT-PCR require invasive biopsy or termination of precious model organisms.

### SUMMARY

Among the various aspects of the present disclosure is the provision of compositions of imaging agents for use in detecting, monitoring, and evaluating ACE2 expression patterns, as well as the use of detected ACE2 expression patterns to diagnose, associated diseases, disorders, and conditions.

Briefly, therefore, the present disclosure is directed to compositions and methods to detect, monitor, and evaluate conditions associated with ACE2 upregulation or overexpression. The compositions and methods of the present disclosure may also be used to determine a prognosis and/or select a treatment for a subject diagnosed with COVID-19.

In one aspect, the present disclosure is directed to an imaging agent comprising an ACE2 binding peptide, a radiolabel, and a chelator. In some aspects, the ACE2 binding peptide may include a linear DX600 peptide or a cyclized DX600 peptide. In some aspects, the ACE2 binding peptide may include an amino acid sequence, Gly-Asp-Tyr-Ser-His-Cys-Ser-Pro-Leu-Arg-Tyr-Tyr-Pro-Trp-Trp-Lys-Cys-Thr-Tyr-Pro-Asp-Pro-Glu-Gly-Gly-Gly (SEQ ID NO: 1). In some aspects, the ACE2 binding peptide may also include one or more chemical modifications that confers resistance to proteolysis. In some aspects, the amino acid sequence may further include one or more conservative substitutions. In some aspects, the radiolabel may include any one of ²H (D or deuterium), ³H (T or tritium), ¹¹C, ¹³C, ¹⁴C, ⁶⁴Cu, ⁶⁷Cu, ¹⁷⁷Lu, ¹³N, ¹⁵N, ¹⁵O, ¹⁷O, ¹⁸O, ¹⁸F, ⁸⁹Sr, ³⁵S, ¹⁵³Sm, ³⁶Cl, ⁸²Br, ⁷⁵Br, ⁷⁶Br, ⁷⁷Br, ¹²³I, ¹²⁴I, ¹²⁵I, ¹³¹I, ¹¹¹In, ⁶⁷Ga, ⁶⁸Ga, ¹⁷⁷Lu, ¹⁸⁶Re, ¹⁸⁸Re, ²⁰¹Tl, ⁹⁹mTc, ⁹⁰Y, ⁸⁹Zr, ²²⁵Ac, ²¹³Bi, ²¹²Pb, ²²⁷Th, ¹⁵⁰Gd, ¹⁵²Gd, ¹⁵³Gd, ¹⁵⁴Gd, ¹⁵⁵Gd, ¹⁵⁶Gd, ¹⁵⁷Gd, ¹⁵⁸Gd, ¹⁶⁰Gd, ²²³Ra, oxygen-15 water, nitrogen-13 ammonia, [⁸²Rb] rubidium-82 chloride, [¹¹C], [¹¹C] ²⁵B-NBOMe, [¹⁸F] Altanserin, [¹¹C] Carfentanil, [¹¹C] DASB, [¹¹C] DTBZ, [¹⁸] Fluoropropyl-DTBZ, [¹¹C] ME@HAPTHI, [¹⁸F] Fallypride, [¹⁸F] Florbetaben, [¹⁸F] Flubatine, [¹⁸F] Fluspidine, [¹⁸F] Florbetapir, [¹⁸F] or [¹¹C] Flumazenil, [¹⁸F] Flutemetamol, [¹⁸F] Fluorodopa, [¹⁸F] Desmethoxyfallypride, [¹⁸F] Mefway, [¹⁸F] MPPF, [¹⁸F] Nifene, Pittsburgh compound B, [¹¹C] Raclopride, [¹⁸F] Setoperone, [¹⁸F] or [¹¹C] N-Methylspiperone, [¹¹C] Verapamil, [¹¹C] Martinostat, Fludeoxyglucose (¹⁸F)(FDG)-glucose analogue, [¹¹C] Acetate, [¹¹C] Methionine, [¹¹C] Choline, [¹⁸] Fluciclovine, [¹⁸F] Fluorocholine, [¹⁸F] FET, [¹⁸F] FMISO, [¹⁸F] 3′-fluoro-3′-deoxythymidine, [⁶⁸Ga] DOTA-pseudopeptides, [⁶⁸Ga] PSMA, and [¹⁸] Fluorodeoxysorbitol (FDS). In some aspects, the radiolabel may include any one of ⁶⁴Cu, ⁶⁸Ga, and ¹⁸F. In some aspects, the chelator may include any one of NHS-MAG3, MAG3, DTPA, 3p-C-NE3TA, 3p-C-NOTA, 3p-C-DE4TA, ATSM, triazamacrocyclic ligands (e.g. NOTA (1,4,7-triazacyclononane-N,N′,N″-triacetic acid) and NODAGA (1,4,7-triazacyclononane,1-glutaric acid-4,7-acetic acid)), tetraazamacrocyclic ligands (e.g., DOTA (1,4,7,10-tetraazacyclododecane-1,4,7,10-tetraacetic acid), DOTA-NHS, pSCN-Bn-DOTA, pNH2-Bn-DOTA, TETA (1,4,8,11-tetraazacyclotetradecane-1,4,8,11-tetraacetic acid, TETA-octreotide (OC), DOTAGA (1,4,7,10-tetraazacyclododecane,1-(glutaric acid)-4,7,10-triacetic acid)), hexaazamacrobicyclic cage-type ligands (e.g., Sarcophogine chelators), cross-bridged tetraamine ligands (e.g., CB-TE2A (4,11-bis(carboxymethyl)-1,4,8,11-tetraazabicyclo[6.6.2]hexadecane)), 6-Hydrazinopridine-3-carboxylic acid (Hynic), NHS-Hynic, and 2,2′,2″-(10-(2-((2-(2,5-dioxo-2,5-dihydro-1H-pyrrol-1-yl)ethyl)amino)-2-oxoethyl)-1,4,7,10-tetraazacyclododecane-1,4,7-triyl)triacetic acid (Maleimido-mono-amide-DOTA). In some aspects, the chelator may be NODAGA (1,4,7-triazacyclononane,1-glutaric acid-4,7-acetic acid), NOTA (1,4,7-triazacyclononane-N,N′,N″-triacetic acid) or DOTA (1,4,7,10-tetraazacyclododecane-1,4,7,10-tetraacetic acid). In some aspects, the chelator may be conjugated to the ACE2 binding peptide and the chelator is radiolabeled. In some aspects, the imaging agent is a PET or SPECT imaging agent selected from ⁶⁴Cu-NODAGA-DX600, ⁶⁴Cu-NOTA-DX600, or ⁶⁸Ga-DOTA-DX600. The imaging agent detects an ACE2 expression profile.

In another aspect, the present disclosure is directed to a method of detecting an ACE2 expression profile in a subject. The method includes administering to the subject any imaging agent described above and detecting the imaging agent. Detecting the imaging agent includes imaging the subject using a detection method that includes at least one of positron emission tomography (PET) imaging, single-photon emission computed tomography (SPECT) imaging, mass spectrometry, gamma imaging, magnetic resonance imaging (MRI), magnetic resonance spectroscopy imaging, fluorescence spectroscopy imaging, CT imaging, ultrasound imaging, photoacoustic imaging, and X-ray imaging.

In an additional aspect, the present disclosure is directed to a method of selecting a treatment for an ACE2 associated disease for a subject. The method includes administering the imaging agent described above to the subject, detecting the imaging agent, and selecting a treatment based on the detected imaging agent. Detecting the imaging agent further includes transforming the detected imaging agent into an ACE2 expression profile. Selecting a treatment based on the detected imaging agent further includes selecting a treatment for the subject if the ACE2 expression rate is elevated above a threshold rate. Selecting a treatment based on the detected imaging agent may also include selecting a treatment for the subject if ACE2 expression is detected within a subject's lungs. The ACE2 associated disease is a disease, disorder, or condition is selected from the group consisting of: a cardiovascular disorder, a renal pathophysiology, diabetes, a lung disorder, an ACE2-associated coronavirus infection, and a cancer. The cardiovascular disorder may include at least one of reduced cardiac contractility, increased fibrosis, cardiac hypertrophy, myocardial fibrosis, pulmonary congestion, cardiac conduction disturbances, heart failure, and blood pressure dysregulation. The renal pathophysiology may include at least one of renal damage, glomerulosclerosis, and albuminuria. The lung disorder may include at least one of pulmonary hypertension, sarcoidosis, idiopathic pulmonary fibrosis, and acute respiratory distress syndrome. The ACE2-associated coronavirus infection may be an infection by at least one of a severe acute respiratory syndrome (SARS)-CoV virus, a human CoV-NL63 virus, and a severe acute respiratory syndrome coronavirus clade 2 (SARS-CoV-2) virus.

In another additional aspect, the present disclosure is directed to a pharmaceutical composition that includes any of the imaging agents described above.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a bar graph comparing SARS-CoV-2 outcomes between patients with and without cancer.

FIG. 1B is a graph comparing the proportions of patients with critical outcomes from COVID-19 for patients with and without cancer.

FIG. 1C is a bar graph comparing SARS-CoV-2 outcomes of non-cancer patients and cancer patients undergoing various forms of cancer therapy.

FIG. 1D is a graph comparing the proportions of patients with critical outcomes from COVID-19 for non-cancer patients and cancer patients undergoing various forms of cancer therapy.

FIG. 2A is a graph summarizing ACE2 expression values by tissue, across male and female volunteers' specimens. Organs at-risk for damage and failure from the virus are generally highly expressing ACE2, including intestine, kidney, heart, and lung.

FIG. 2B is a scatter plot summarizing tissue-specific fold changes in ACE2 expression with respect to sex (male/female).

FIG. 2C is a scatter plot summarizing tissue-specific fold changes in ACE2 expression with respect to age (old/young).

FIG. 3A is a schematic diagram illustrating SARS-CoV-2 and cell entry through binding and internalization via spike protein interaction with ACE2.

FIG. 3B is a diagram showing the chemical structure of linear peptide modified with NODAGA (blue) to stably chelate the copper-64 radiometal for PET imaging.

FIG. 3C is a diagram showing the chemical structure of cyclized peptide (disulfide bond, red) modified with NODAGA (blue) to stably chelate the copper-64 radiometal for PET imaging.

FIG. 3D contains a radio-chromatogram of the radiolabeled peptide of FIG. 3C for quality control reporting. The agent is rapidly labeled to >98% purity

FIG. 3E contains an absorbance chromatogram of the radiolabeled peptide of FIG. 3C for quality control reporting. The agent is rapidly labeled to >98% purity.

FIG. 3F is a bar graph summarizing the results of a specificity and binding analysis of the cyclized peptide of FIG. 3C at 120 minutes of incubation with hACE2-expressing or control vehicle cells.

FIG. 3G is a graph summarizing the proportion of membrane-bound and internalized fractions of the ⁶⁴Cu-HZ-20 of FIG. 3C exposed to 1.5E5 human-ACE2 expressing (blue) and control vehicle (red) HEK293T cells. Rapid uptake and internalization at 37° C. encouraged in vivo evaluation of this molecule.

FIG. 3H is a graph summarizing the proportion of internalized fractions of the ⁶⁴Cu-HZ-20 of FIG. 3C exposed to 1.5E5 human-ACE2 expressing (blue) and control vehicle (red) HEK293T cells. Rapid uptake and internalization at 37° C. encouraged in vivo evaluation of this molecule.

FIG. 4A contains a series of dynamic PET images obtained at various times after administration of 15 MBq ⁶⁴Cu-labeled linear peptide of FIG. 3B (HZ-19); h denotes heart, b denotes bladder, and k denotes kidney.

FIG. 4B contains a series of dynamic PET images obtained at various times after administration of 15 MBq ⁶⁴Cu-labeled cyclized peptide of FIG. 3C (HZ-20); h denotes heart, b denotes bladder, and k denotes kidney.

FIG. 4C is a graph summarizing a quantitative analysis by percent injected activity per mL of tissue (% IA/mL) by organ for the linear ⁶⁴Cu-labeled peptide of FIG. 3B.

FIG. 4D is a graph summarizing a quantitative analysis by percent injected activity per mL of tissue (% IA/mL) by organ for the cyclized ⁶⁴Cu-labeled peptide of FIG. 3C.

FIG. 4E is a schematic illustration showing organs of clearance (liver, kidneys, and bladder) and tumors (hACE2− left; hACE2+ right) of the hACE2±Dual Xenograft Model.

FIG. 4F is a dynamic PET image obtained 18 hours after the administration of the linear ⁶⁴Cu-labeled peptide of FIG. 3B.

FIG. 4G is a dynamic PET image obtained 18 hours after the administration of the cyclized ⁶⁴Cu-labeled peptide of FIG. 3C.

FIG. 5A is a histogram summarizing the ex vivo biodistribution in tissues and tumors at 18 h post administration of the linear ⁶⁴Cu-labeled peptide of FIG. 3B (Linear) and the cyclized ⁶⁴Cu-labeled peptide of FIG. 3C (Cyclized).

FIG. 5B contains maps of the autoradiographic signal from hACE2-positive (right) and negative (left) HEK293T tumors 18 hours after administration of the cyclized ⁶⁴Cu-labeled peptide of FIG. 3C (scale bar=1 mm).

FIG. 5C contains coronal slice images of PET images obtained from a non-tumor-bearing wild-type animal immediately after injection (left) and one hour after injection of the cyclized ⁶⁴Cu-labeled peptide of FIG. 3C.

FIG. 5D is a histogram summarizing the ex vivo biodistribution in tissues 18 hours after administration of the cyclized ⁶⁴Cu-labeled peptide of FIG. 3C to a non-tumor-bearing wild-type animal.

FIG. 6A is a schematic diagram illustrating SARS-CoV-2 and cell entry through binding and internalization via spike protein interaction with ACE2, DX600 and radiolabeled DX600 analogs may interface the penetration of SARS-CoV-2 into host cells by specifically binding to ACE2 receptor.

FIG. 6B is a diagram showing the chemical structure of ACE2 targeting peptides modified with a chelator (NODAGA or DOTA) to stably chelate a radiolabel (copper-64 or gallium-68) radiometal for PET imaging.

FIG. 6C is a graph summarizing the results of a saturation binding assay of ^{64/Nat}Cu-HZ20 over HEK293-hACE2 and HEK293-WT (non-transduced) at 4° C.

FIG. 6D is a graph summarizing the results of a saturation binding assay of HZ20.

FIG. 6E is a graph summarizing the time-dependent binding and internalization of ⁶⁴Cu-HZ20 over HEK293-hACE2 cells at 37° C.

FIG. 7A is an in vivo PET image of Dual xenografts (Left: HEK293-WT, Right: HEK293-hACE2) imaged with ⁶⁴Cu-HZ20 at 18 h post injection.

FIG. 7B is a graph summarizing the quantitative analysis of dynamic ⁶⁴Cu-HZ20 imaging of the dual xenografts of FIG. 7A, kidneys, heart, and liver.

FIG. 7C is a series of images showing ex vivo autoradiographic imaging of collected dual xenografts and H&E staining after PET imaging at 18 h post injection.

FIG. 7D contains a series of images showing micro-PET/CT imaging of ⁶⁸Ga-HZ20 in ACE2 expressing HepG2 tumor-bearing mice at 2 h post-injection (right), shown compared with the blocking control (left). In the blocking group, ⁶⁸Ga-HZ20 was co-injected with 50 mg/kg of DX600. The upper and lower images are MIP images and cross-sectional images of the mice, respectively.

FIG. 7E is a graph comparing the SUVmax of Micro-PET imaging at 2 h post-injection. Heart, liver, lung, kidney, muscle, and tumor were selected for both groups (n=4) and statistical significance was observed for the tumor in comparison to the control group, 0.099±0.0065 vs 0.024±0.0041 (****: P<0.0001).

FIG. 7F contains a series of images showing the immunohistochemistry results of representative mouse tissues.

FIG. 8A is a graph summarizing dynamic changes of the SUVmax ratio of selected-organ-to-lung at 7 time points (5 min, 14 min, 23 min, 32 min, 40 min, 90 min, 180 min) for ⁶⁸Ga-HZ20 distribution in the oropharynx, nasal mucosa, and eyes of volunteer subjects; these organs are representative of organs exposed to virus entry.

FIG. 8B is a graph similar to the graph of FIG. 8A for the breast, gallbladder, and testis of volunteer subjects.

FIG. 8C is a graph similar to the graph of FIG. 8A for the renal cortex, spleen, liver, and pancreas of volunteer subjects.

FIG. 8D is a graph showing the rank ordering of organ ACE2 expression in different organs indicated by SUVmax. The average SUVmax from 20 healthy volunteers at 90 min scans are shown in the columns (M: male only, F: female only). The SUVmax of the organs from the recovered subjects at 90 min scan is indicated with a star.

FIG. 9A is a representative PET image of ⁶⁸Ga-HZ20 radioactivity uptake in a 38-year-old male volunteer (#009) at 90 min post-injection. The MIP and transverse image showed that the nasal mucosa (1), gallbladder (2), small intestine (4) showed moderate radioactivity accumulation, and the renal cortex (3) and testis (5) showed high accumulation.

FIG. 9B is a representative PET image of ⁶⁸Ga-HZ20 radioactivity uptake in a 34-year-old female volunteer (#012) at 90 min post-injection. The MIP and transverse image showed that the nasal mucosa (1), breast (2), and gallbladder (3) showed moderate accumulation, and the renal cortex (4) and the corpus luteum of left ovary (5) showed high accumulation.

FIG. 9C is a series of images showing the immunohistochemistry analysis of ACE2 expression in normal human organs (10×).

FIG. 10A is a transverse T2WI+fs MR scan of ⁶⁸Ga-HZ20 uptake in the ovaries of a female volunteer taken on the 21st day of menstrual cycle at 2 h post-injection of ⁶⁸Ga-HZ20 on the same day. Both ovary foci localized in the corpus luteum (red arrow), the stroma of the ovary showed slight uptake, and no uptake was observed in immature follicles (blue arrow).

FIG. 10B is a PET image obtained from the ovary of the subject imagined in FIG. 10A under similar conditions.

FIG. 10C is a fusion image of the images shown in FIGS. 10A and 10B.

FIG. 10D is a transverse T2WI+fs MR scan of ⁶⁸Ga-HZ20 uptake in the ovaries of a female volunteer taken on the 22nd day of menstrual cycle at 2 h post-injection of ⁶⁸Ga-HZ20 on the same day. Both ovary foci localized in the corpus luteum (red arrow), the stroma of the ovary showed slight uptake, and no uptake was observed in immature follicles (blue arrow).

FIG. 10E is a PET image obtained from the ovary of the subject imagined in FIG. 10D under similar conditions.

FIG. 10F is a fusion image of the images shown in FIGS. 10D and 10E.

FIG. 11A is a graph comparing the SUVmax values of the breast tissues of young (<50 years old) and old (≥50 years old) volunteers at 90 min.; the breast tissues of young volunteers had significantly higher uptake.

FIG. 11B is a graph comparing the SUVmax values of the ovary tissues of young (<50 years old) and old (≥50 years old) volunteers at 90 min.; no significant differences in uptake were observed.

FIG. 11C is a graph comparing the SUVmax values of the testis tissues of young (<50 years old) and old (≥50 years old) volunteers at 90 min; no significant differences in uptake were observed.

FIG. 11D is a graph comparing the SUVmax values of the gallbladder tissues of male and female volunteers at 90 min.; no significant differences in uptake were observed.

FIG. 11E is a graph comparing the SUVmax values of the nasal mucosa tissues of male and female volunteers at 90 min.; no significant differences in uptake were observed.

FIG. 11F is a graph comparing the SUVmax values of the renal cortex tissues of male and female volunteers at 90 min.; no significant differences in uptake were observed.

FIG. 12A shows a full-body PET image of a 38-year-old male infected with COVID-19 and recovered at 90 min. post-injection.

FIG. 12B shows axial CT (upper) and PET (lower) images of the nasal tissues obtained from the subject of FIG. 12A under similar conditions; nasal mucosa is denoted by arrows.

FIG. 12C shows axial CT (upper) and PET (lower) images of the lung tissues obtained from the subject of FIG. 12A under similar conditions; lungs are denoted by arrows.

FIG. 12D shows axial CT (upper) and PET (lower) images of the testis tissues obtained from the subject of FIG. 12A under similar conditions; testes are denoted by arrows.

FIG. 12E is a series of MIP maps of the patient of FIG. 12A (left), a male healthy volunteer (center), and a female healthy volunteer (right). The renal cortex showed lower uptake of ⁶⁸Ga-HZ20 than healthy male and female volunteers. The SUVmax of renal cortex was lower than healthy volunteers (SUVmax of 11.59 vs 22.18±1.78 for the left kidney, 18.40 vs 25.67±1.39 for the right kidney). The gallbladder and testis showed higher uptake than healthy volunteers (SUVmax of 4.32 vs 2.14±0.39 for gallbladder, 8.79 vs 4.51±0.54 for the right testis, 8.65 vs 4.58±0.40 for the left testis).

FIG. 13A is a MALDI-TOF mass spectrum of DX600, Cal. M-H- [C141H186N35O40S2-]=3073.31, m/z=3073.26.

FIG. 13B is a MALDI-TOF mass spectrum of DOTA-DX600, Cal. M-H- [C155H210N39O46S2-]=3417.47, m/z=3417.48.

FIG. 14A is a graph summarizing a saturation binding assay of ^{64/Nat}Cu-HZ20 over HEK293-hACE2 and HEK293-WT (non-transduced) at 37° C.

FIG. 14A is a graph summarizing a saturation binding assay of ^{64/Nat}CU-HZ20 over HEK293-hACE2 and HEK293-WT (non-transduced) at 37° C.

FIG. 14B is a graph summarizing a binding assay of DX600.

FIG. 14C is a graph of hACE2 and WT expression; hACE2 expression specifically determined the accumulation of ⁶⁴Cu-HZ20 over HEK293-hACE2 and HEK293-WT cells for 2 h incubation at 37° C.

FIG. 14D is a graph summarizing a stability analysis of ⁶⁸Ga-HZ20 over time in 0.01 M PBS solution at 37° C.

FIG. 15A is a series of graphs showing dynamic PET imaging of the dual xenografts using ⁶⁴Cu-HZ20 for HEK293-hACE2 (right) and HEK293-WT (left) for 60 min after dose administration.

FIG. 15B is a static PET image of hACE2 transgenic (Tg) mice at 2 h post injection.

FIG. 15C contains a comparison of static PET images of hACE2 Tg (left) and wild type (right) animals at 24 post injection.

FIG. 15D is a graph summarizing a quantitative analysis of static ⁶⁴Cu-HZ20 imaging of hACE2 Tg and wild type mice, 24 h post injection.

FIG. 16A is a graph of the HPLC signal of HZ20 monitored by UV channel, t_{R}=9.93 min.

FIG. 16B is a graph of HPLC signal of ⁶⁸Ga-HZ20 before purification monitored by radioactivity channel, t_{R}=10.38 min. The calculated radio-chemical purity was 91.98%.

FIG. 16C is a graph of HPLC signal of ⁶⁸Ga-HZ20 after purification monitored by radioactivity channel, t_{R}=10.34. The calculated radio-chemical purity was 98.54%. The HPLC was eluted with water-CH₃CN system (Phase A: 0.1% TFA H₂O; Phase B: 0.1% TFA CH₃CN) using gradient elution (0-5 min: 20% B; 5-10 min: 20%-80% B; 10-12 min: 80% B; 12-15 min: 80%-20% B) at a flow rate of 1.0 mL/min.

FIG. 17 is a graph summarizing the results of body weight monitoring following DX600 exposure conducted in mice for 25 days. The experiment was carried out in 10 mice at 4-weeks of age. The mice were either injected with DX600 of 200 μg/200 μL or 0.9% normal saline of the same volume. The injection was repeated on day 0, day 4, day 11, day 18 and day 25, and the weight of the mice was recorded. The results showed that there was no significant difference between the experimental group (n=5, weight from 26.5±5.2 g to 41.2±3.1 g) and the control group (n=5, weight from 25.4±5.6 g to 40.4±4.7 g).

FIG. 18A is a graph summarizing the bio-distribution in male mice at 5, 30, 60, 120, 240 min post-injection (n=4).

FIG. 18B is a graph summarizing the bio-distribution results in female mice at 5, 30, 60, 120, 240 min post-injection (n=4). Kidneys received the highest radioactivity uptake and the radioactivity could also accumulate in the spleen, stomach, intestine, and several other tissues. The normal animal body distribution was used for preliminary analysis and reference before translation research. Data are shown as percent injected activity per gram of tissue (% IA/g).

FIG. 19A is a graph comparing the maximum standardized uptake values (SUVmax) of tumor, heart, liver, lung, kidney, and muscle tissues between the experimental group and the blocking control group at 60 min based on micro-PET imaging and quantitative analysis of ⁶⁸Ga-HZ20 in HepG2 tumor-bearing mice. DX600 was applied as a blocking agent with a dose of 50 mg/kg.

FIG. 19B is a graph comparing the maximum standardized uptake values (SUVmax) of tumor, heart, liver, lung, kidney, and muscle tissues between the experimental group and the blocking control groups of FIG. 19A at 120 min.

FIG. 19C is a graph comparing the maximum standardized uptake values (SUVmax) of tumor, heart, liver, lung, kidney, and muscle tissues between the experimental group and the blocking control groups of FIG. 19A at 180 min.

FIG. 19D is a graph summarizing the ratio of the uptake of tumor-to-normal-organ between the experimental group and blocking group at each time point based on data from FIGS. 19A, 19B, and 19C.

FIG. 19E is a comparison of images between the experimental group and blocking control under the same scale, with controls shown on the top row. ⁶⁸Ga-HZ20 has a specific uptake in HepG2 tumors expressing human ACE2, which is significantly higher than that of the blocking group (****: 0.00001<P≤0.0001, *****:P≤0.00001).

FIG. 20A is a graph summarizing SUVmax results in 20 volunteers obtained at 5 min.

FIG. 20B is a graph summarizing SUVmax results in 20 volunteers obtained at 14 min.

FIG. 20C is a graph summarizing SUVmax results in 20 volunteers obtained at 23 min.

FIG. 20D is a graph summarizing SUVmax results in 20 volunteers obtained at 32 min.

FIG. 20E is a graph summarizing SUVmax results in 20 volunteers obtained at 40 min.

FIG. 20F is a graph summarizing SUVmax results in 20 volunteers obtained at 180 min.

FIG. 20G is a graph summarizing dynamic changes of SUVmax within the oropharynx, nasal mucosa, and eye tissues.

FIG. 20H is a graph summarizing dynamic changes of SUVmax within the breast, gallbladder, testis, and uterus tissues.

FIG. 20I is a graph summarizing dynamic changes of SUVmax within the renal cortex, spleen, liver, pancreas, and lung tissues.

FIG. 21 is a bar graph comparing SUVmax in a variety of tissues of COVID-19 patients and healthy volunteers. The uptake in most organs of the recovered COVID-19 patient at 90 min was not within the confidence interval of healthy volunteers, except for the right renal medulla, left adrenal gland, and ileum. There is a considerable difference between the recovered patient and healthy volunteers.

FIG. 22 contains a series of images showing dynamic PET images of a female volunteer obtained after injection of 134 MBq ⁶⁸Ga-HZ20.

FIG. 23A is a dynamic PET image of the conjunctiva of a male volunteer with SUVmax of 1.5; specific radioactivity uptake in eyes, especially conjunctiva, although at a medium level was observed in agreement with previously published results. Conjunctiva was indicated by arrows.

FIG. 23B is a dynamic PET image of the conjunctiva of a female volunteer with SUVmax of 1.8, demonstrating similar results to those of FIG. 23A. Conjunctiva was indicated by arrows.

FIG. 24 is a graph comparing left and right breast SUVmax between young and old groups at different scanning times. The breast uptake in young females was significantly higher than that of old females at multiple time points (14 min, 23 min, 32 min, and 40 min), but this difference tended to drop at 90 min and 180 min (**: 0.001<P≤0.01).

FIG. 25 is a graph comparing radioactivity uptake in symmetrical organs, quantified as SUVmax difference between the right and left organ (right minus left) at 90 min imaging. The confidence interval of the eyes (−0.09 to 0.10), lungs (−0.02 to 0.10), and testis (−0.38 to 0.25) which cover zero showed little difference, while renal cortex (3.77 to 10.90) and renal medulla (1.90 to 11.84) which over zero showed an observable higher SUVmax in the right organ.

FIG. 26A shows an illustration of HZ20 binding ACE2 and Variants. The enzymatic carboxypeptidase cleavage site is inhibited with nM affinity by HZ20 in the cyclical conformation. Radioconjugation has not altered binding affinity. Chelate-HZ20 shown in surface energy minimized human ACE2 (HPEPDock).

FIG. 26B shows an illustration of HZ20 binding ACE2 and Variants. We annotate high frequency (allele count) mutations in the binding pocket of ACE2 inhibitory-peptide and spike protein receptor-binding domain (RBD). Structures rendered in ChimeraX v1.3 from cryo-EM crystal structures of ACE2/SARS-CoV-2 RBD, PDB: 6VW1.

FIG. 27 is a schematic overview of PG-PEM DAR Restoration. Digital autoradiography is a key target engagement validation technique, that often suffers from low resolution, image blur, and noise. We apply an iterative penalized MLEM approach to estimate the point-spread function of the image, without requiring calibration, to restore the image while simultaneously separating background and signal elements. The result is increased signal to noise, resolution, and data utility.

FIG. 28A shows a representative bone biopsy segmented for calcified tissue (white).

FIG. 28B shows raw DAR data acquired and overlaid from an 8 μm thick cryosection.

FIG. 28C shows the raw data of FIG. 28B subjected to the PG-PEM iterative algorithm of FIG. 27; the algorithm qualitatively improves DAR quality.

FIG. 28D is a graph of CNR for the image of FIG. 28B (raw) and FIG. 28C (PG-PEM), demonstrating reduced background noise with PG-PEM (higher is better).

FIG. 28E is a graph of effective resolution for the image of FIG. 28B (raw) and FIG. 28C (PG-PEM), demonstrating that PG-PEM improves tissue-signal resolution (lower is better).

FIG. 28F is a graph of fusion for the image of FIG. 28B (raw) and FIG. 28C (PG-PEM), demonstrating that PG-PEM improves registration of anatomical optical and radiographic modalities (percent total activity associated with the bone surface; higher is better).

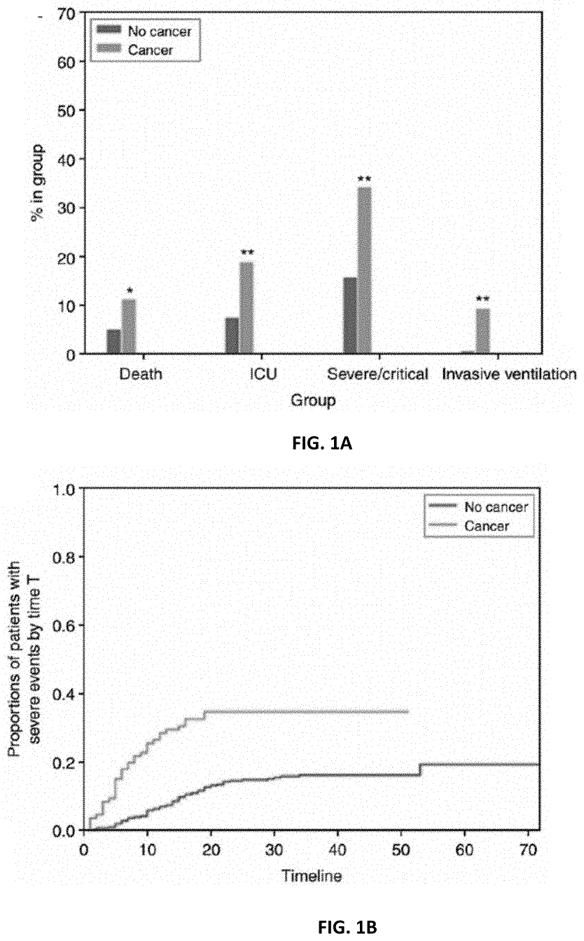

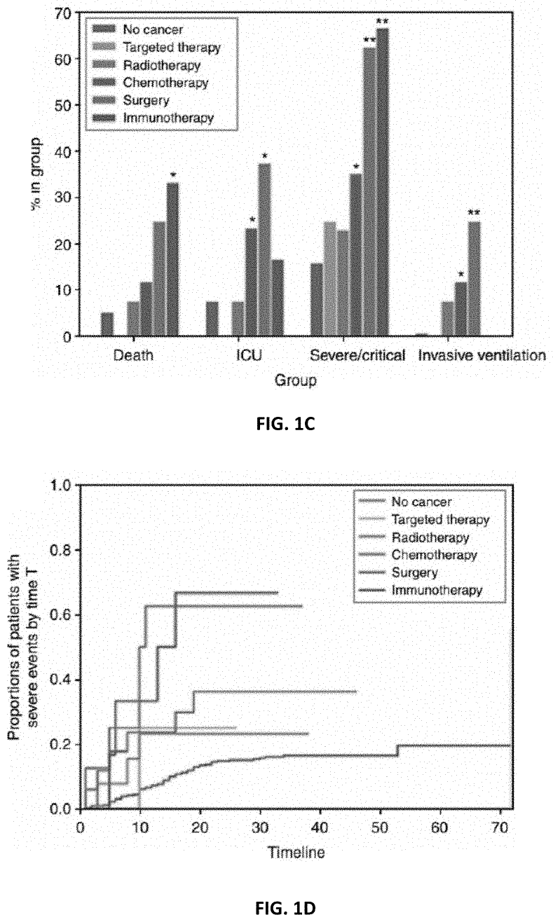

### DETAILED DESCRIPTION

The present disclosure is based, at least in part, on the discovery that an ACE2 binding peptide adapted as an imaging agent including, but not limited to, a PET probe can detect ACE2 expression profiles in mouse or other disease research models, as well as in human tissues.

Molecular imaging has emerged as a non-invasive approach to quantitatively monitor biochemical processes occurring inside organisms in real time. Positron emission tomography (PET) utilizes radioligands to interrogate disease-relevant biomarkers or pathways providing quantitative spatial distribution of physiologically relevant targets with exquisite sensitivity. ⁶⁴Cu and ⁶⁸Ga-labeled peptides that specifically targeting ACE2 are described herein. The disclosed labeled peptides are useful in a variety of clinical contexts including, but not limited to, characterizing the effects of ACE2 expression on SARS-CoV-2 infection and COVID-19 severity.

In various aspects, a high affinity, high specificity imaging agent is disclosed that is capable of quantitatively delineating human ACE2 (hACE2) expression in vivo to enable the longitudinal study of the receptor under experimental conditions. In some aspects, the disclosed imaging agent is based on an ACE2 inhibiting peptide, including, but not limited to, DX600, that includes the amino acid sequence Gly-Asp-Tyr-Ser-His-Cys-Ser-Pro-Leu-Arg-Tyr-Tyr-Pro-Trp-Trp-Lys-Cys-Thr-Tyr-Pro-Asp-Pro-Glu-Gly-Gly-Gly (SEQ ID NO: 1). Without being limited to any particular theory, DX-600 previously demonstrated specific inhibition of hACE2 (Kᵢ=2.8 nM).

In various aspects, the ACE2 inhibiting peptide is labeled with a contrast agent to produce the imaging agent. The contrast agent is selected depending on the imaging method to be used with the imaging agent. In some aspects, the ACE2 inhibiting peptide is labeled with a radiolabel including, but not limited to copper-64 to produce an imaging agent compatible with an imaging method including, but not limited to, PET imaging. In some aspects, the ACE2 inhibiting peptide is derivatized with a chelate including, but not limited to, NODAGA (1,4,7-triazacyclononane,1-glutaric acid-4,7-acetic acid) to facilitate radiolabeling the ACE2 inhibiting peptide.

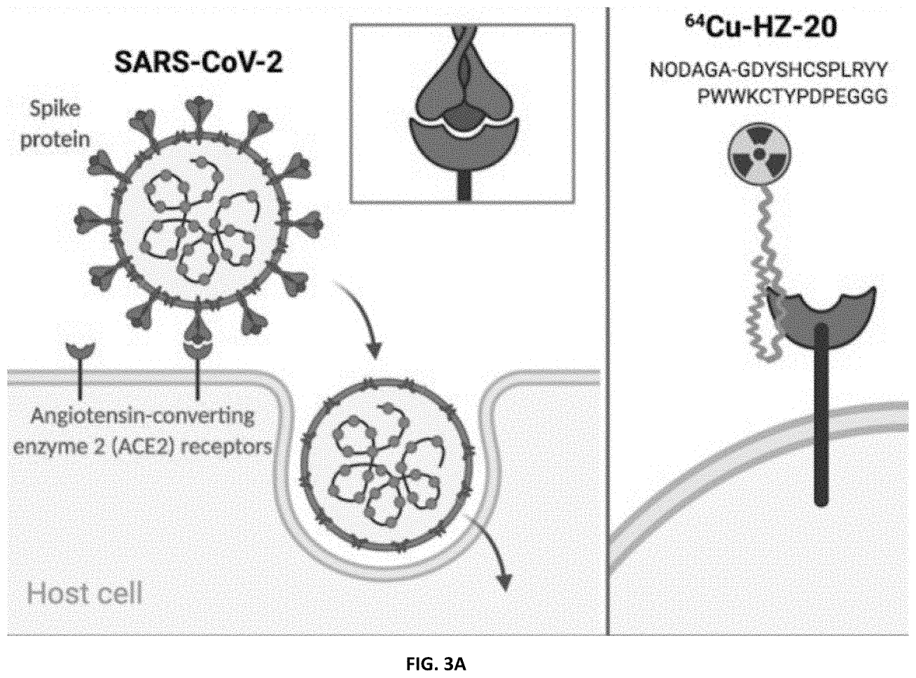

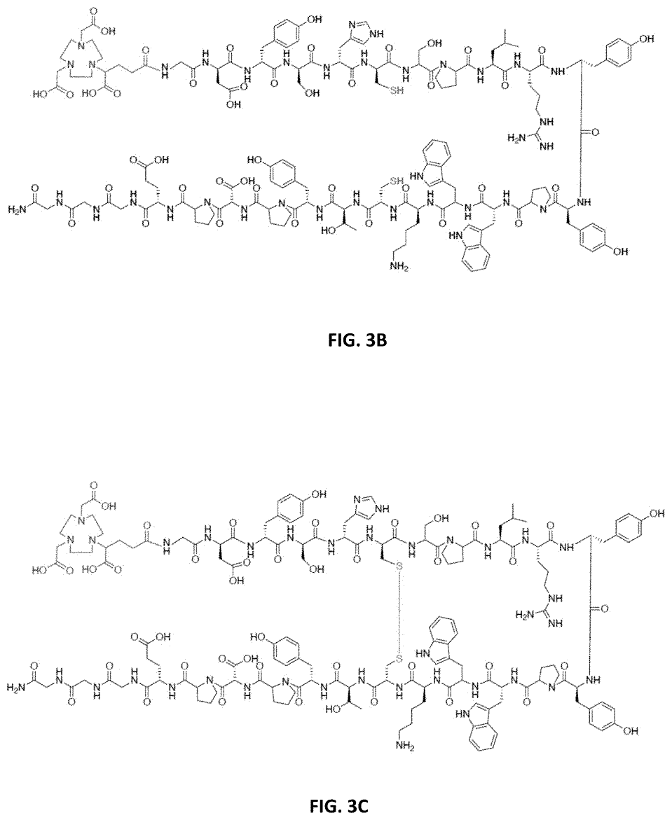

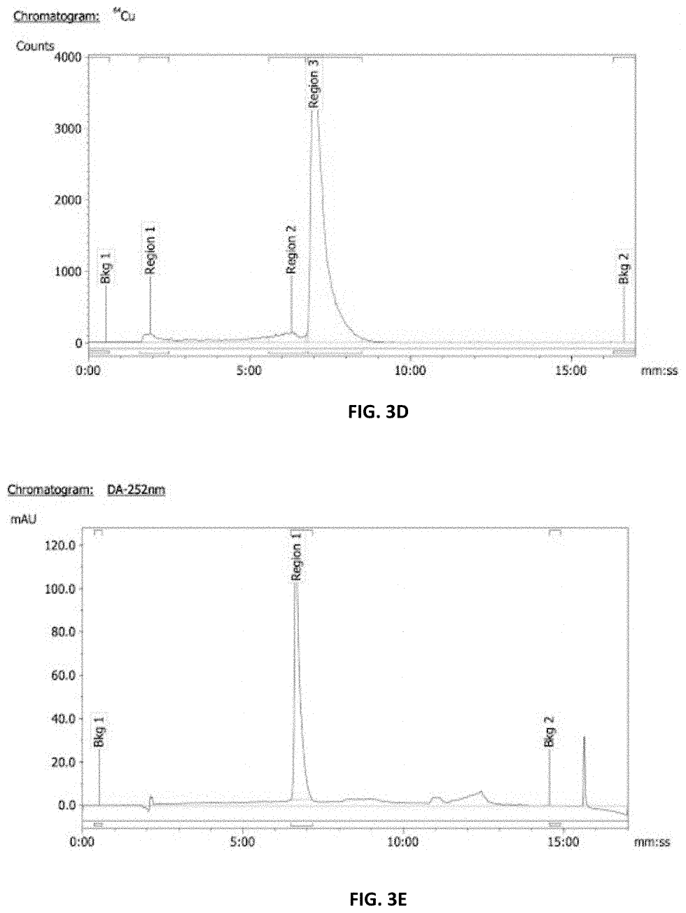

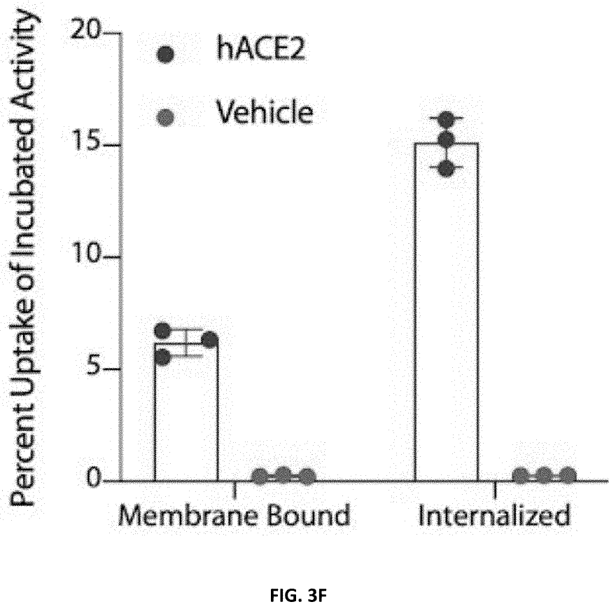

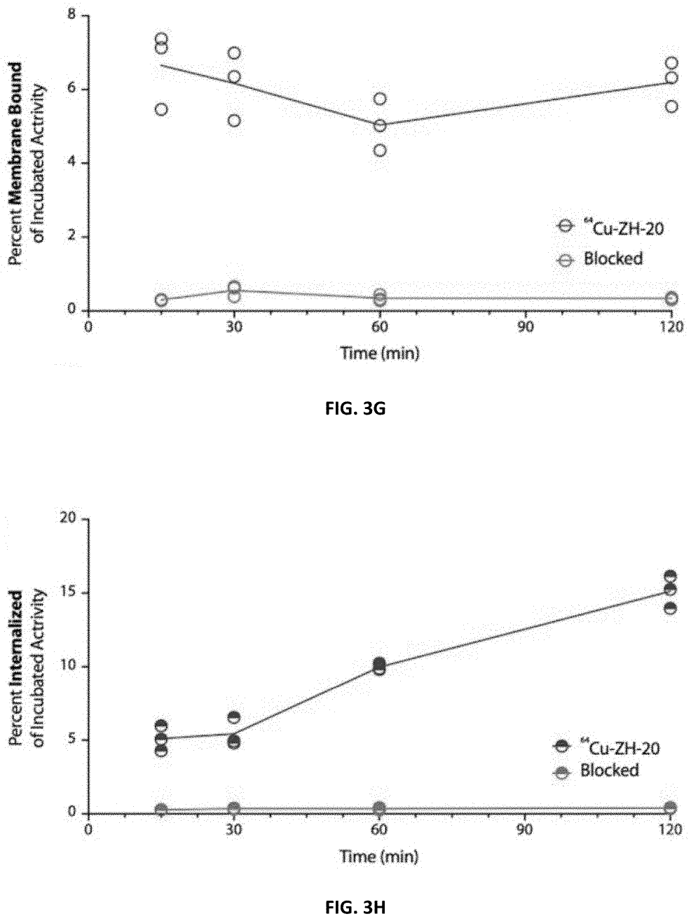

In some aspects, the radiotracer comprises DX600 peptide derivatized with NODAGA, referred to herein as NODAGA-DX600. In one aspect, the NODAGA-DX600 is provided in a linear form, referred to herein as ZH-19, shown illustrated in FIG. 3B. In another aspect, the NODAGA-DX600 is provided in a cyclized form, referred to herein as ZH-20, shown illustrated in FIG. 3C. In other aspects, NODAGA-DX600 is radiolabeled using ⁶⁴Cu to produce the radiotracer ⁶⁴Cu-NODAGA-DX600. In various aspects, ⁶⁴Cu-NODAGA-DX600 may be provided in a linear form, referred to herein as ⁶⁴Cu-ZH-19, or in a cyclized form, referred to herein as ⁶⁴Cu-ZH-20. In one aspect, the ⁶⁴Cu-ZH-20 is a sensitive imaging agent configured to specifically delineate the distribution of human ACE2 receptors in vivo.

In various aspects, the disclosed imaging agents (e.g., ⁶⁴Cu-NODAGA-DX600) make possible the specific and sensitive detection of ACE2 up-regulated at various locations associated with ACE2-associated diseases or disorders including, but not limited to, lungs, kidneys, liver, and heart in animal models. The biological characterization of ACE2 expression in these models may support the implementation of the PET imaging agents disclosed herein for clinical use.

As described herein, an ACE2 binding peptide, DX600 may be adapted as an imaging agent including, but not limited to, a positron emission tomography (PET) radiotracer that is used to detect changes in ACE2 expression associated with the development or progression of an ACE2-related disorder or disease.

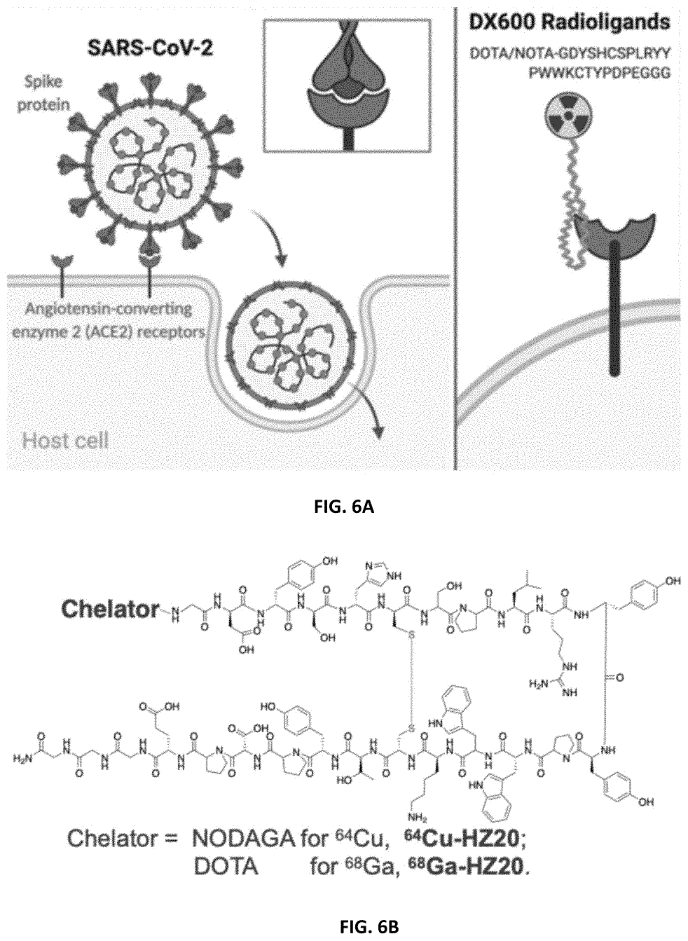

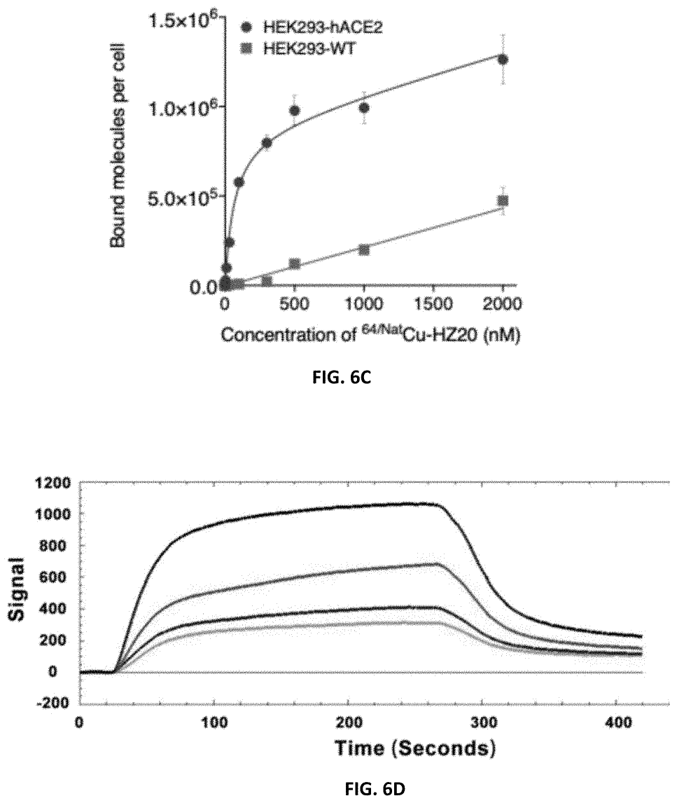

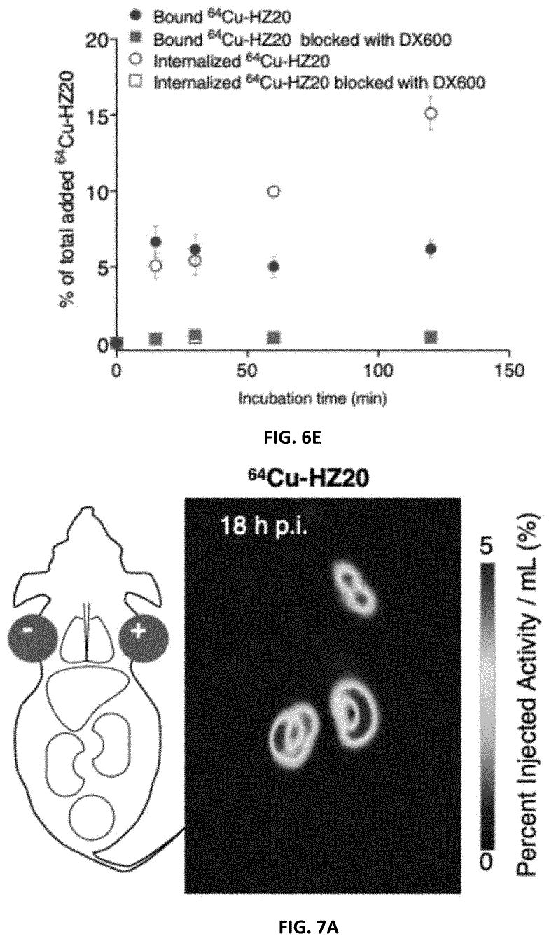

As described in the Examples below, the ACE2-specific PET radioligands displayed excellent imaging and pharmacokinetic properties. Preclinical model systems were used to quantify ACE2 expression in vivo using ⁶⁸Ga- and ⁶⁴Cu-labeled analogs of the ACE2-targeting DX600 peptide: ⁶⁸Ga-HZ20 and ⁶⁴Cu-HZ20, respectively (FIG. 6A). The human-specific ACE2 inhibiting cyclic peptide DX600 (⁶⁸Ga-HZ20) was advanced to the clinic for PET/CT imaging and organ-based standardized uptake value (SUV analysis) of 20 healthy volunteers. A patient who had recovered from SARS-CoV-2 infection was included, and demonstrated greater ⁶⁸Ga-HZ20 SUVmax (a standardized measure of tracer accumulation). Complementing pathological analyses, these intriguing results demonstrate that radiolabeled HZ20 analogs have substantial value in quantifying the ACE2 distribution in the entire body, as well as monitoring the transient upregulation of ACE2 expression to guide patient management and evaluate therapeutic intervention of COVID-19 patients.

Angiotensin-Converting Enzyme 2 (ACE2)

The present disclosure provides the first imaging probe available for ACE2 detection. As described herein, the specificity and sensitivity of imaging ACE2 expression profiles using the disclosed ACE2 imaging agent have been well characterized in pre-clinical studies.

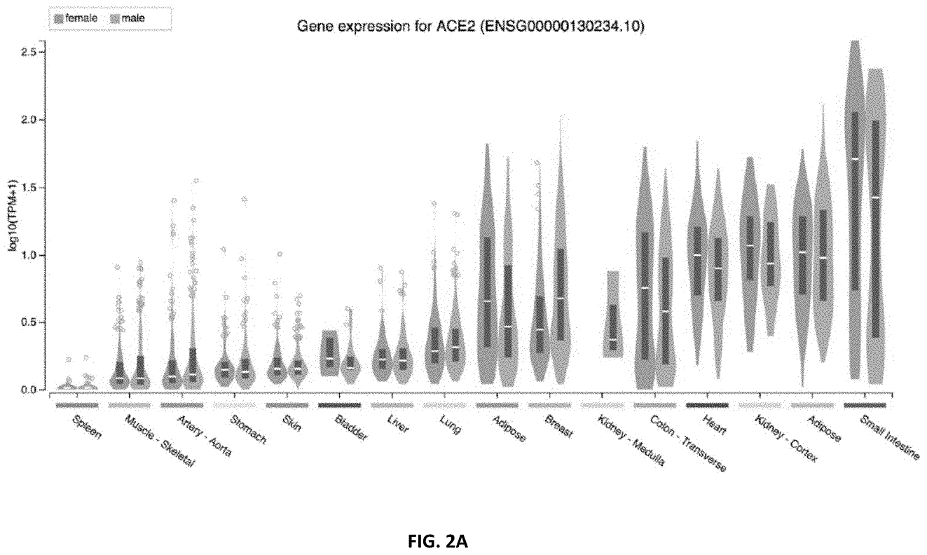

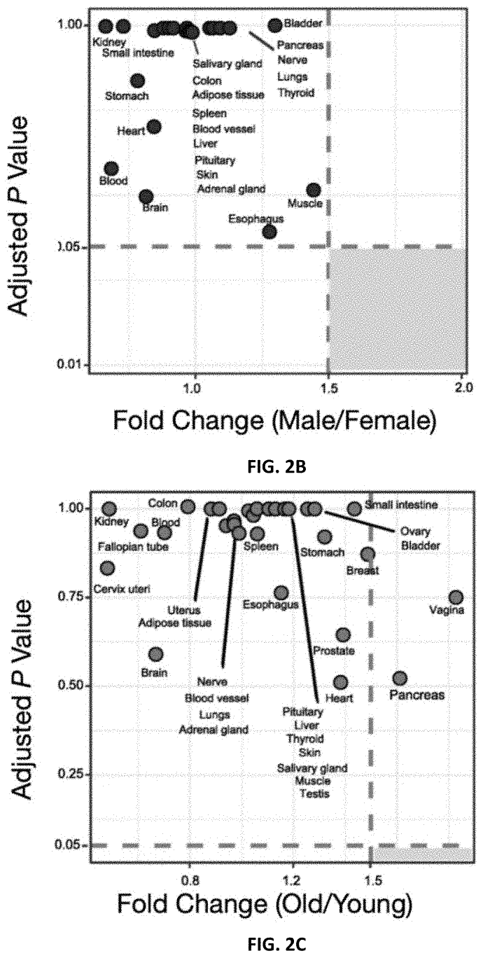

Angiotensin-converting enzymes ACE and ACE2, are key members of the renin angiotensin system. ACE2 is a trans-membrane mono-carboxypeptidase with an extracellular catalytic domain to remove a single amino acid from the octapeptide angiotensin II to generate angiotensin1-7. It is widely expressed in tissues including: heart, vessels, gut, lung, kidney, testis, and brain (FIG. 2A) and has cardiovascular protective functions. However, in the case of SARS-CoV-2 and COVID-19, it is unknown if the virus exploits the receptor for infection alone or also blocks these beneficial effects. Analysis of archival genetic databases has been performed for age and sex determinants of ACE2 expression that may explain why both the elderly and men have increased mortality from COVID-19. Large differences in tissue-specific ACE2 expression but these changes are not significant.

Imaging Agent/ACE2 Probes

In various aspects, the structure and use of ACE2-specific molecular probes or imaging agents are disclosed herein.

In various aspects, the imaging agent was developed through the conjugation of a targeting peptide for positron emission tomography (PET) imaging of humans and of various mouse models used to study ACE2 associated diseases, disorders, and conditions. Non-limiting examples of ACE2 associated diseases, disorders, and conditions include cardiac disease, lung disease, kidney disease, and coronavirus infections.

In various aspects, the imaging agent specifically binds to human (h)ACE2. The cyclized peptide (see FIG. 3C) was derived from a screen for hACE2 enzyme inhibitors and displays low nanomolar potency. However, the imaging agent does not itself inhibit SARS-CoV (2005) cell entry. The parent compound DX-600 specifically inhibits hACE2 (Kᵢ=2.8 nM). This DX-600 lead was derivatized with a chelate for radiolabeling of reduced-linear peptide (see FIG. 3B) or cyclized peptide (see FIG. 3C), referred to herein as ⁶⁴Cu-HZ-19 and ⁶⁴Cu-HZ-20, respectively. Radiolabeling is rapid and approaches stoichiometric purity through gentle heating at pH 5 (see FIG. 3D and FIG. 3E).

In various aspects, the disclosed imaging agents provides a powerful tool for the sensitive and specific detection of ACE2 to track ACE2 expression in various pathological conditions and lay the foundation for new approaches for the diagnosis and treatment of ACE2-mediated processes using imaging modalities including, but not limited to, PET imaging.

In various aspects, the imaging agent can comprise a radiolabel, an ACE2 binding peptide, and/or a chelator. The imaging agent can be formulated to be detected by any method of imaging known in the art. Non-limiting examples of imaging devices and methods suitable for use in detecting the disclosed imaging agent include PET imaging in which the imaging agent is a PET imaging agent or tracer. Other non-limiting examples of imaging devices and methods suitable for use in detecting the disclosed imaging agent include SPECT imaging, mass spectrometry, MRI, NMR imaging, fluorescence spectroscopy, computed tomography (CT) imaging, ultrasound imaging, photoacoustic imaging, and X-ray imaging.

The present disclosure provides a robust imaging probe for ACE2 detection. In various aspects, the imaging agents are ACE2 imaging agents based on the DX600 peptide.

In some aspects, the imaging agent is a positron emission tomography (PET) and single-photon emission computed tomography (SPECT) imaging probe that includes an ACE2 binding peptide (e.g., DX600) to image the expression profile of ACE2 cell surface proteins in support of in vitro cell studies as well as in vivo studies using a range of animal disease models as well as human subjects. In some aspects, the peptide can be conjugated with a macrocyclic chelator for radiolabeling with various radionuclides or grafted on a nanostructure with controlled physicochemical properties. In various aspects, the conjugation strategy and compositions of developed imaging probes may be optimized for enhanced binding affinity and improved in vivo pharmacokinetics.

In some aspects, PET imaging may be performed in animal models or human subjects administered the disclosed imaging agent to non-invasively track the specific cell population expressing ACE2 at site-of-interest. In other aspects, fluorescent or other tags may be conjugated on the peptide to track the probe for non-radioactive studies.

In various aspects, human or animal subjects administered the disclosed imaging agent may be imaged using any of the imaging modalities described herein to obtain an ACE2 expression profile, to diagnose an ACE2 associated disease or disorder, to monitor the progression of an ACE2 associated disease or disorder, to select a treatment for an ACE2 associated disease or disorder, to screen a candidate treatment for an ACE2 associated disease, and/or to evaluate a clinical outcome of subject administered a treatment for an ACE2 associated disease. In some aspects, the disclosed imaging agents can be useful for the evaluation of the treatment for ACE2 associated diseases, disorders, or conditions to optimize the treatment strategy and to improve the therapeutic efficacy.

In various aspects, the imaging agent may be a biocompatible imaging agent. As such, the imaging agent can be stored in or prepared in a buffer of a physiologically relevant pH. For example, the pH can be about 1 to 3 (e.g., for stomach); about 4 to 7 (e.g., for small intestine); about 7-8.5 (e.g., for large intestine); about 7.4 (e.g., for blood pH); about 7.35 (e.g., for CSF); or about 5 to 6 (e.g., for urine pH). As another example, the pH can be about 1; about 1.5; about 2; about 2.5; about 3; about 3.5; about 4; about 4.5; about 5; about 5.5; about 6; about 6.5; about 6.6; about 6.7; about 6.8; about 6.9; about 7; about 7.1; about 7.2; about 7.3; about 7.4; about 7.5; about 8; or about 8.5.

ACE2 Binding Peptide

In various aspects, the imaging agent comprises an ACE2 binding peptide. The ACE2 binding peptide may be any peptide with ACE2 binding activity without limitation. By way of non-limiting example, the ACE2 binding peptide can be an ACE2 non-competitive antagonist peptide. By way of another non-limiting example, the ACE2 binding peptide can be a cyclized ACE2 binding peptide.

In some aspects, the binding peptide may be a linear DX600 peptide or a cyclized DX600 peptide. In various aspects, the ACE2 binding peptide comprises: the amino acid sequence Gly-Asp-Tyr-Ser-His-Cys-Ser-Pro-Leu-Arg-Tyr-Tyr-Pro-Trp-Trp-Lys-Cys-Thr-Tyr-Pro-Asp-Pro-Glu-Gly-Gly-Gly (SEQ ID NO: 1), a sequence deriving from SEQ ID NO: 1 by one or more chemical modifications that confer resistance to proteolysis; or a sequence deriving from SEQ ID NO: 1 by one or more conservative substitutions.

The ACE2 binding peptide as described herein can comprise an amino acid length of about 4 amino acids to about 200 amino acids or about 4 amino acids to about 50 amino acids. For example, the ACE2 binding peptide can comprise an amino acid length of no more than 4 amino acids; 5 amino acids; 6 amino acids; 7 amino acids; 8 amino acids; 9 amino acids; 10 amino acids; 11 amino acids; 12 amino acids; 13 amino acids; 14 amino acids; 15 amino acids; 16 amino acids; 17 amino acids; 18 amino acids; 19 amino acids; 20 amino acids; 21 amino acids; 22 amino acids; 23 amino acids; 24 amino acids; 25 amino acids; 26 amino acids; 27 amino acids; 28 amino acids; 29 amino acids; 30 amino acids; 31 amino acids; 32 amino acids; 33 amino acids; 34 amino acids; 35 amino acids; 36 amino acids; 37 amino acids; 38 amino acids; 39 amino acids; 40 amino acids; 41 amino acids; 42 amino acids; 43 amino acids; 44 amino acids; 45 amino acids; 46 amino acids; 47 amino acids; 48 amino acids; 49 amino acids; 50 amino acids; 51 amino acids; 52 amino acids; 53 amino acids; 54 amino acids; 55 amino acids; 56 amino acids; 57 amino acids; 58 amino acids; 59 amino acids; 60 amino acids; 61 amino acids; 62 amino acids; 63 amino acids; 64 amino acids; 65 amino acids; 66 amino acids; 67 amino acids; 68 amino acids; 69 amino acids; 70 amino acids; 71 amino acids; 72 amino acids; 73 amino acids; 74 amino acids; 75 amino acids; 76 amino acids; 77 amino acids; 78 amino acids; 79 amino acids; 80 amino acids; 81 amino acids; 82 amino acids; 83 amino acids; 84 amino acids; 85 amino acids; 86 amino acids; 87 amino acids; 88 amino acids; 89 amino acids; 90 amino acids; 91 amino acids; 92 amino acids; 93 amino acids; 94 amino acids; 95 amino acids; 96 amino acids; 97 amino acids; 98 amino acids; 99 amino acids; 100 amino acids; 101 amino acids; 102 amino acids; 103 amino acids; 104 amino acids; 105 amino acids; 106 amino acids; 107 amino acids; 108 amino acids; 109 amino acids; 110 amino acids; 111 amino acids; 112 amino acids; 113 amino acids; 114 amino acids; 115 amino acids; 116 amino acids; 117 amino acids; 118 amino acids; 119 amino acids; 120 amino acids; 121 amino acids; 122 amino acids; 123 amino acids; 124 amino acids; 125 amino acids; 126 amino acids; 127 amino acids; 128 amino acids; 129 amino acids; 130 amino acids; 131 amino acids; 132 amino acids; 133 amino acids; 134 amino acids; 135 amino acids; 136 amino acids; 137 amino acids; 138 amino acids; 139 amino acids; 140 amino acids; 141 amino acids; 142 amino acids; 143 amino acids; 144 amino acids; 145 amino acids; 146 amino acids; 147 amino acids; 148 amino acids; 149 amino acids; 150 amino acids; 151 amino acids; 152 amino acids; 153 amino acids; 154 amino acids; 155 amino acids; 156 amino acids; 157 amino acids; 158 amino acids; 159 amino acids; 160 amino acids; 161 amino acids; 162 amino acids; 163 amino acids; 164 amino acids; 165 amino acids; 166 amino acids; 167 amino acids; 168 amino acids; 169 amino acids; 170 amino acids; 171 amino acids; 172 amino acids; 173 amino acids; 174 amino acids; 175 amino acids; 176 amino acids; 177 amino acids; 178 amino acids; 179 amino acids; 180 amino acids; 181 amino acids; 182 amino acids; 183 amino acids; 184 amino acids; 185 amino acids; 186 amino acids; 187 amino acids; 188 amino acids; 189 amino acids; 190 amino acids; 191 amino acids; 192 amino acids; 193 amino acids; 194 amino acids; 195 amino acids; 196 amino acids; 197 amino acids; 198 amino acids; 199 amino acids; or 200 amino acids. Recitation of each of these discrete values is understood to include ranges between each value. Recitation of each of a range is understood to include discrete values within the range.

Radiolabel

In some aspects, the imaging agent, as described herein, comprises a radiolabel (also known as a radionuclide). Any radiolabeling process known in the art may be used to produce the disclosed imaging agent without limitation. One non-limiting example of a suitable radiolabeling method is described in Fani et al. Theranostics 2012; 2(5):481-501. doi:10.7150/thno.4024, which is incorporated by reference herein in its entirety. Except as otherwise noted herein, therefore, the process of the present disclosure can be carried out in accordance with such processes.

One embodiment of the present disclosure provides for a radiolabeled peptide. According to another embodiment, the radiolabeled compound can be an imaging agent.

References herein to “radiolabeled” include a compound where one or more atoms are replaced or substituted by an atom having an atomic mass or mass number different from the atomic mass or mass number typically found in nature (i.e., naturally occurring). One non-limiting exception is ¹⁹F, which allows detection of a molecule that contains this element without enrichment to a higher degree than what is naturally occurring. Compounds carrying the substituent ¹⁹F may thus also be referred to as “labelled” or the like. The term radiolabeled may be interchangeably used with “isotopically-labelled”, “labelled”, “isotopic tracer group”, “isotopic marker”, “isotopic label”, “detectable isotope”, or “radioligand”.

In one embodiment, the compound comprises one or more radiolabeled groups.

Non-limiting examples of suitable radiolabel groups include: ²H (D or deuterium), ³H (T or tritium), ¹¹C, ¹³C, ¹⁴C, ⁶⁴Cu, ⁶⁷Cu, ¹⁷⁷Lu, ¹³N, ¹⁵N, ¹⁵O, ¹⁷O, ¹⁸O, ¹⁸F, ⁸⁹Sr, ³⁵S, ¹⁵³Sm, ³⁶Cl, ⁸²Br, ⁷⁵Br, ⁷⁶Br, ⁷⁷Br, ¹²³I, ¹²⁴I, ¹²⁵I, ¹³¹I, ¹¹¹In, ⁶⁷Ga, ⁶⁸Ga, ¹⁷⁷Lu, ¹⁸⁶Re, ¹⁸⁸Re, ²⁰¹Tl ^{99m}Tc, ⁹⁰Y, or ⁸⁹Zr. It is to be understood that an isotopically labeled compound needs only to be enriched with a detectable isotope to, or above, the degree which allows detection with a technique suitable for the particular application, e.g., in a detectable compound labeled with ¹¹C, the carbon-atom of the labeled group of the labeled compound may be constituted by ¹²C or other carbon-isotopes in a fraction of the molecules. The radionuclide that is incorporated in the radiolabeled compounds will depend on the specific application of that radiolabeled compound. For example, “heavy” isotope-labeled compounds (e.g., compounds containing deuterons/heavy hydrogen, heavy nitrogen, heavy oxygen, heavy carbon) can be useful for mass spectrometric and NMR-based studies. As another example, for in vitro labelling or in competition assays, compounds that incorporate ³H, ¹⁴C, or ¹²⁵I can be useful. For in vivo imaging applications ¹¹C, ¹³C, ¹⁸F, ¹⁹F, ¹²⁰I, ¹²³I, ¹³¹I, ⁷⁵Br, or ⁷⁶Br can generally be useful. In one embodiment, the radiolabel is ⁶⁴Cu.

Non-limiting examples of imaging agents comprising a radiolabel can comprise Oxygen-15 water, Nitrogen-13 ammonia, [⁸²Rb] Rubidium-82 chloride, [¹¹C], [¹¹C] 25B-NBOMe, [¹⁸] Altanserin, [¹¹C] Carfentanil, [¹¹C] DASB, [¹¹C] DTBZ, [¹⁸] Fluoropropyl-DTBZ, [¹¹C] ME@HAPTHI, [¹⁸] Fallypride, [¹⁸] Florbetaben, [¹⁸] Flubatine, [¹⁸] Fluspidine, [¹⁸] Florbetapir, [¹⁸] or [¹¹C] Flumazenil, [¹⁸] Flutemetamol, [¹⁸] Fluorodopa, [¹⁸] Desmethoxyfallypride, [¹⁸] Mefway, [¹⁸] MPPF, [¹⁸] Nifene, Pittsburgh compound B, [¹¹C] Raclopride, [¹⁸] Setoperone, [¹⁸] or [¹¹C] N-Methylspiperone, [¹¹C] Verapamil, [¹¹C] Martinostat, Fludeoxyglucose (¹⁸F)(FDG)-glucose analogue, [¹¹C] Acetate, [¹¹C] Methionine, [¹¹C] Choline, [¹⁸] Fluciclovine, [¹⁸] Fluorocholine, [¹⁸] FET, [¹⁸] FMISO, [¹⁸] 3′-fluoro-3′-deoxythymidine, [⁶⁸Ga] DOTA-pseudopeptides, [⁶⁸Ga] PSMA, or [¹⁸] Fluorodeoxysorbitol (FDS).

Chelator

As described herein, radionuclides can be chelated by any method known in the art. The disclosed imaging agents may be chelated using any known method without limitation. Non-limiting examples of suitable chelation methods are described in Anderson et al., Cancer Biother Radiopharm. 2009 August; 24(4): 379-393; and Stockholf et al., Pharmaceuticals (Basel). 2014 April; 7(4): 392-418, the contents of each of which are incorporated by reference herein in its entirety. Except as otherwise noted herein, therefore, the process of the present disclosure can be carried out in accordance with such processes.

Non-limiting examples of suitable chelators include NHS-MAG3, MAG3, DTPA, 3p-C-NE3TA, 3p-C-NOTA, 3p-C-DE4TA, ATSM, triazamacrocyclic ligands (e.g. NOTA (1,4,7-triazacyclononane-N,N′,N″-triacetic acid) and NODAGA (1,4,7-triazacyclononane,1-glutaric acid-4,7-acetic acid)), tetraazamacrocyclic ligands (e.g., DOTA (1,4,7,10-tetraazacyclododecane-1,4,7,10-tetraacetic acid), DOTA-NHS, pSCN-Bn-DOTA, pNH₂—Bn-DOTA, TETA (1,4,8,11-tetraazacyclotetradecane-1,4,8,11-tetraacetic acid, TETA-octreotide (OC), DOTAGA (1,4,7,10-tetraazacyclododecane,1-(glutaric acid)-4,7,10-triacetic acid)), hexaazamacrobicyclic cage-type ligands (e.g., Sarcophogine chelators), cross-bridged tetraamine ligands (e.g., CB-TE2A (4,11-bis(carboxymethyl)-1,4,8,11-tetraazabicyclo[6.6.2]hexadecane)), 6-Hydrazinopridine-3-carboxylic acid (Hynic), NHS-Hynic, or 2,2′,2″-(10-(2-((2-(2,5-dioxo-2,5-dihydro-1H-pyrrol-1-yl)ethyl)amino)-2-oxoethyl)-1,4,7,10-tetraazacyclododecane-1,4,7-triyl)triacetic acid (Maleimido-mono-amide-DOTA). For example, chelators for a radiolabel (e.g., ⁶⁴Cu) can be any of those known in the art (e.g., a macrocyclic chelator). In one aspect, the chelator is NODAGA (1,4,7-triazacyclononane,1-glutaric acid-4,7-acetic acid).

Nanoparticle

As described herein, a radiolabel can be doped in or on a nanoparticle, or a radiolabel can be conjugated to a nanoparticle.

The imaging agent, as described herein can comprise any nanoparticle known in the art suitable for use as an imaging agent without limitation. Nanoparticles for use in molecular probes and imaging agents are well known. Non-limiting examples of suitable nanoparticles are described in Chen et al., Molecular Imaging Probes for Cancer Research, 2012, the contents of which are incorporated by reference in their entirety.

Labeling of nanoparticles is well known. Non-limiting examples of methods of labeling nanoparticles are described in Yongjian Liu, Michael J Welch, Nanoparticles labeled with positron emission nuclides: advantages, methods, and applications, Bioconjugate Chemistry, 2012, 23, 671-682; and in Stockholf et al., Pharmaceuticals (Basel). 2014 April; 7(4): 392-418, the contents of each of which are incorporated by reference herein in its entirety. Except as otherwise noted herein, therefore, the process of the present disclosure can be carried out in accordance with such processes.

For example, a nanoparticle can be a nanocluster or any other type of nanostructures including organic, inorganic, or lipid nanostructures.

As another example, the nanoparticle can comprise Au or Cu. As another example, the nanoparticle can comprise iron oxide, gold, gold nanoclusters (AuNC), gold nanorods (AuNR), copper (Cu), quantum dots, carbon nanotubes, carbon nanohorn, gadolinium (Gd), dendrimers, dendrons, polyelectrolyte complex (PEC) nanoparticles, calcium phosphate nanoparticles, perfluorocarbon nanoparticles (PFCNPs), or lipid-based nanoparticles, such as liposomes and micelles.

Linker

Described herein are linkers used to attach peptides to a portion of an imaging agent (e.g., a core, a nanoparticle, a radiolabel, a chelator, another peptide). A linker can be any composition used for conjugation, for example to a nanoparticle or chelator.

A linker group can be any linker group suitable for use in an imaging agent. Linker groups for imaging agents (e.g., molecular probes) are well known. Non-limiting examples of suitable linkers are described in Werengowska-Ciećwierz et al., Advances in Condensed Matter Physics, Vol. 2015 (2015); and in Chen et al., Curr Top Med Chem. 2010; 10(12): 1227-1236), the contents of each of which are incorporated by reference herein in its entirety. Except as otherwise noted herein, therefore, the processes of the present disclosure can be carried out in accordance with such processes.

For example, the linker can conjugate a nanoparticle to an ACE2 binding peptide. For example, the ACE2 binding peptide can be covalently attached to the linker. For example, the linker can comprise a poly(ethylene glycol) (PEG) derivative. As another example, the linker can comprise PEG, TA-PEG-Maleimide, TA-PEG-OMe, or TA-PEG. As another example, a linker can comprise an isothiocyanate group, a carboxylic acid or carboxylate groups, a dendrimer, a dendron, Fmoc-protected-2,3-diaminopropanoic acid, ascorbic acid, a silane linker, minopropyltrimethoxysilane (APTMS), or dopamine. Other covalent coupling methods can use employ the use of 2 thiol groups, 2 primary amines, a carboxylic acid and primary amine, maleimide and thiol, hydrazide and aldehyde, or a primary amine and aldehyde. For example, the linker can be an amide, a thioether, a disulfide, an acetyl-hydrazone group, a polycyclic group, a click chemistry (CC) group (e.g., cycloadditions, for example, Huisgen catalytic cycloaddition; nucleophilic substitution chemistry, for example, ring opening of heterocyclic electrophiles; carbonyl chemistry of the “nonaldol” type, for example, formation of ureas, thioureas, and hydrazones; additions to carbon-carbon multiple bonds, for example, epoxidation and dihydroxylation); or a physical or chemical bond.

Detecting ACE2 Associated Disease, Disorders, or Conditions

As described herein, the present disclosure provides for methods of detecting or imaging ACE2 expression, and/or evaluating or monitoring an ACE2 associated disease, disorder, or condition. Without being limited to any particular theory, ACE2 expression is typically upregulated in ACE2 associated diseases, disorders, or conditions.

ACE2 Associated Diseases, Disorders, or Conditions

An ACE2 associated disease can be any disease, disorder, or condition in which ACE2 is involved, is associated with ACE2, or is an ACE2 mediated syndrome. Non-limiting examples of ACE2 associated diseases, disorders, or conditions include cardiovascular disorders, renal pathophysiology, diabetes, lung disorders, cancers, and coronavirus infections.

Non-limiting examples of ACE2-associated cardiovascular disorders include reduced cardiac contractility, increased fibrosis, cardiac hypertrophy, myocardial fibrosis, pulmonary congestion, cardiac conduction disturbances, heart failure, and blood pressure dysregulation. Non-limiting examples of ACE2-associated renal pathophysiology include renal damage, glomerulosclerosis, and albuminuria. Non-limiting examples of ACE2-associated lung disorders include pulmonary hypertension, sarcoidosis, idiopathic pulmonary fibrosis, and acute respiratory distress syndrome. Non-limiting examples of ACE2-associated coronavirus infections include infections by a severe acute respiratory syndrome (SARS)-CoV virus, a human CoV-NL63 virus, and a severe acute respiratory syndrome coronavirus clade 2 (SARS-CoV-2) virus.

ACE2 Modulating Compounds

In various aspects, the ACE2 associated disorders identified using the disclosed imaging agent and associated methods may be treated using any suitable treatment modality including, but not limited to, administration of an ACE2 modulating compound. The ACE2 modulating compounds, as described herein can be any ACE2 agonist or ACE2 antagonist known in the art without limitation.

In various aspects, ACE2 antagonists include, but are not limited to binding peptides, small molecules, and antibodies. The non-invasive ACE2 imaging techniques made possible using the disclosed imaging agents not only monitor the degree of receptor occupancy to aid in dose selection, but also to determine the therapeutic response in real time.

In one aspect, an ACE2 antagonist is a peptide ACE2 binder including, but not limited to, DX600.

Molecular Engineering

The following definitions and methods are provided to better define the present disclosure and to guide those of ordinary skill in the art in the practice of the present disclosure. Unless otherwise noted, terms are to be understood according to conventional usage by those of ordinary skill in the relevant art.

“Wild-type” refers to a virus or organism found in nature without any known mutation.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

Formulation

The imaging agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Monitoring or Evaluation Methods

Also provided is a process of evaluating or monitoring an ACE2 associated disease, disorder, or condition in a subject in need during administration of a therapeutically effective amount of a therapeutic agent (e.g., an ACE2 modulating compound), so as to substantially inhibit an ACE2 associated disease, disorder, or condition, slow the progress of an ACE2 associated disease, disorder, or condition, or limit the development of an ACE2 associated disease, disorder, or condition.

Methods described herein are generally performed on a subject in need thereof. A subject in need of the evaluation or therapeutic methods described herein can be a subject having, diagnosed with, suspected of having, or at risk for developing an ACE2 associated disease, disorder, or condition. A determination of the need for treatment will typically be assessed by a history and physical exam consistent with the disease or condition at issue. The determination of the need for treatment additionally may be assessed based on the ACE2 expression profile using the ACE2 imaging agent compositions and methods described above. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a therapeutic agent or an imaging agent is, for example, that amount that would cause the desired therapeutic or imaging effect in a subject while minimizing undesired side effects. In various embodiments, an effective amount of a therapeutic agent described herein can substantially inhibit a ACE2 associated disease, disorder, or condition, slow the progress of a ACE2 associated disease, disorder, or condition, or limit the development of an ACE2 associated disease, disorder, or condition.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an imaging or therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to evaluate a ACE2 associated disease, disorder, or condition, substantially inhibit a ACE2 associated disease, disorder, or condition, slow the progress of an ACE2 associated disease, disorder, or condition, or limit the development of an ACE2 associated disease, disorder, or condition.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level or dose level to be used as an imaging agent for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an imaging agent or a therapeutic agent can occur as a single event or over a time course of treatment. For example, an imaging agent or a therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for an ACE2 associated disease, disorder, or condition.

A therapeutic agent can be administered simultaneously or sequentially with another agent, such as an antibiotic, an anti-inflammatory, or another agent. For example, a therapeutic agent can be administered simultaneously with another agent, such as an antibiotic or an anti-inflammatory. Simultaneous administration can occur through administration of separate compositions, each containing one or more of a therapeutic agent, an antibiotic, an anti-inflammatory, or another agent. Simultaneous administration can occur through administration of one composition containing two or more of a therapeutic agent, an antibiotic, an anti-inflammatory, or another agent. A therapeutic agent can be administered sequentially with an antibiotic, an anti-inflammatory, or another agent. For example, a therapeutic agent can be administered before or after administration of an antibiotic, an anti-inflammatory, or another agent.

Administration

The imaging agents as described herein can be administered in a subject in an amount effective to produce images in a variety of imaging modalities, such as PET or SPECT. Administration and calculations of amounts of imaging agents are well known in the art and also described in Long et al., The Chemistry of Molecular Imaging, Wiley, 2014; Saini et al., Spect and MRI Imaging Agents: Brain and Tumor Imaging, Lambert, 2016; Smith et al., Diagnostic Imaging for Pharmacists, APA, 2012). Except as otherwise noted herein, therefore, the process of the present disclosure can be carried out in accordance with such processes.

Agents and compositions described herein can be administered according to methods described herein in a variety of means known to the art. The agents and composition can be used either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

Agents and compositions described herein can be administered in a variety of methods well known in the arts. For example, administration can be parenteral, pulmonary, oral, topical, intradermal, intramuscular, intraperitoneal, intravenous, subcutaneous, intranasal, epidural, ophthalmic, buccal, or rectal administration. As another example, administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

In some aspects, the imaging agent described herein may be administered by IV, and may also be administered by IP or IM. Imaging may commence at any time after administration of the imaging agent, including, but not limited to, about 90 minutes after administration of the imaging agent.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

In various other aspects, the present disclosure is directed to kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to imaging agents, nanoparticles, peptides (e.g., ACE2 binding peptides), chelators, radiolabelled compositions, buffers. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water or sterile saline, each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audiotape, and the like. Detailed instructions may not be physically associated with the kit; instead, a user may be directed to an Internet website specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Characterization of ACE2 Binding by Imaging Agent

To characterize the binding efficacy of imaging agents ⁶⁴Cu-HZ-19 and ⁶⁴Cu-HZ-20, described above, the following experiments were conducted.

⁶⁴Cu-HZ-20 was incubated with HEK293T cells engineered to express the hACE2 (AdV-hACE2) vector or vehicle. As illustrated in FIG. 3F, both membrane-bound and internalized ⁶⁴Cu-HZ-20 was detected in the HEK293T cells engineered to express hACE2 (AdV-hACE2) vector, but not in the vector-expressing HEK293T cell cultures. As illustrated in FIG. 3G and FIG. 3H, membrane-bound and internalized ⁶⁴Cu-HZ-20 was detected within 15 minutes after exposure to the hACE2-expressing cells.

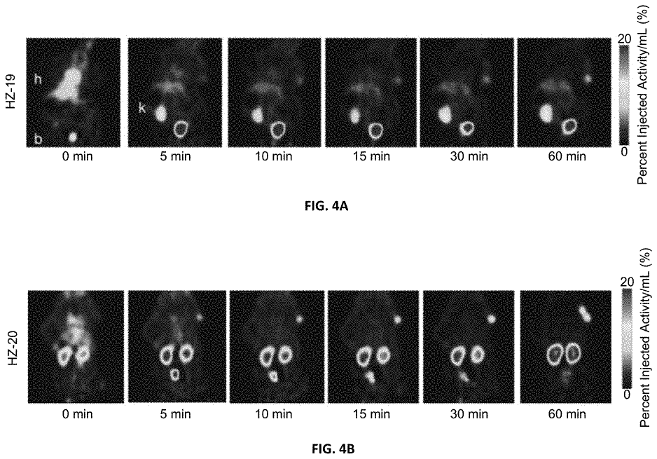

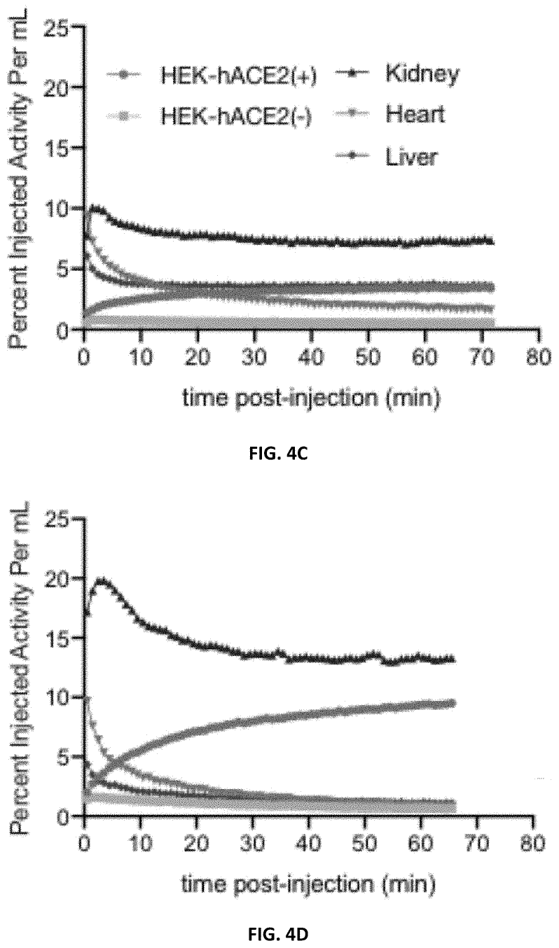

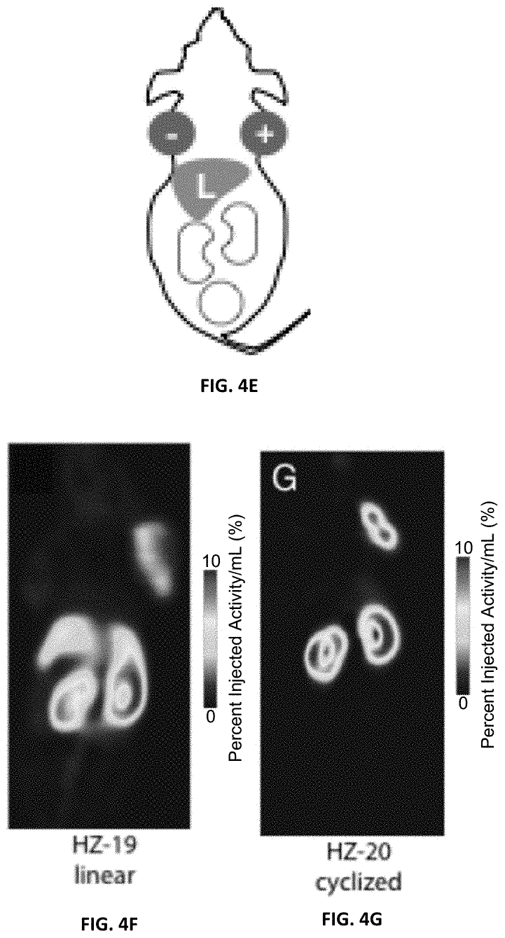

The linear (⁶⁴Cu-HZ-19) and cyclized (⁶⁴Cu-HZ-20) versions of the radiopeptide were evaluated in mice with hACE2-expressing pseudotumors (female R2G2) for early-dynamic and later imaging at 4, 18, and 24 h (see FIG. 4). Clearance from the blood pool for both peptides was rapid, with ⁶⁴Cu-HZ-19 having increased liver retention (see FIG. 4A and FIG. 4C). Greater hACE2 tumor binding was seen with ⁶⁴Cu-HZ-20 and greater kidney clearance (see FIG. 4B and FIG. 4D).

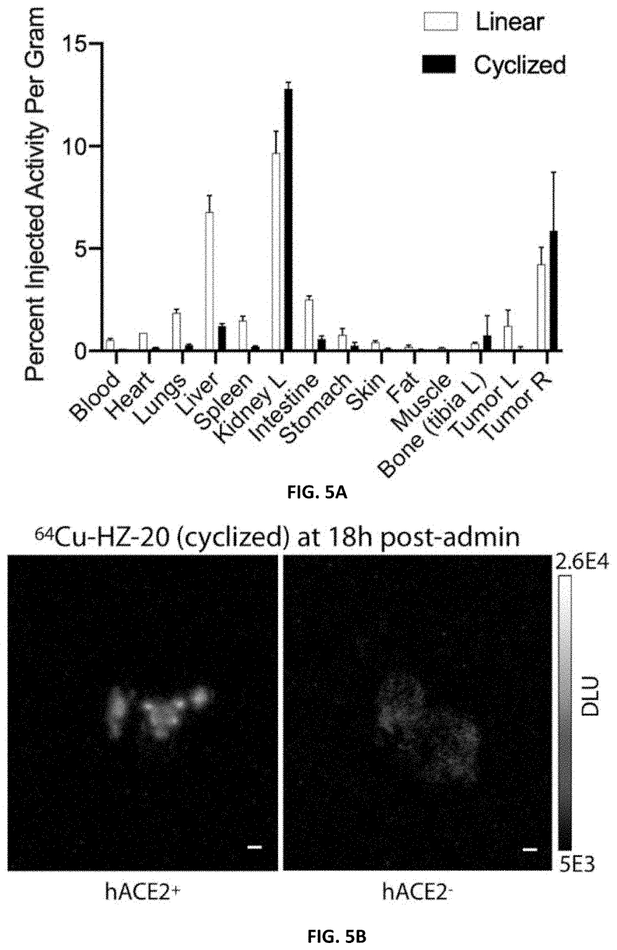

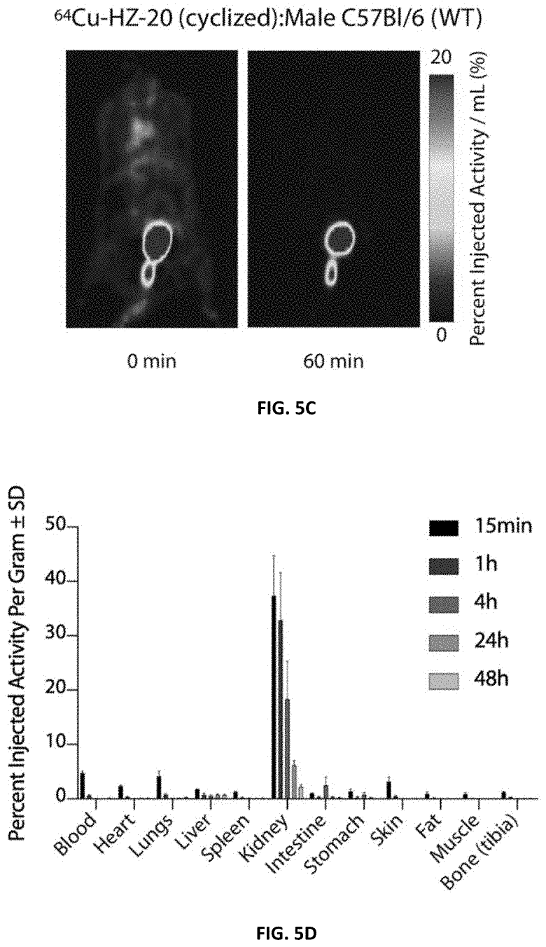

Ex vivo biodistribution analysis of the tracer in these hACE2 expressing (and vehicle control) tumors recapitulated these imaging findings (FIG. 5A). Interestingly, retention of the signal at these later time points is encouraging of the overall utility of ⁶⁴Cu-HZ-20 to delineate hACE2 tissue-level expression as clearance from the blood pool is rapid, and apparent internalization leads to long-lived cell-bound signal (FIG. 3H, FIG. 4B, FIG. 4G, and FIG. 5A). Animals without hACE2 expressing xenografts have limited or very low tissue retention by imaging or organ counting, demonstrating very favorable pharmacokinetic properties of this peptide (FIG. 5C, FIG. 5D, N=3).

### Example 2: Characterization of ACE2 Binding by Imaging Agent

To characterize the molecularly specific positron emission tomography (PET) imaging agents for targeting ACE2 described herein, the following experiments were conducted. PET imaging agents based on DX600, a high affinity ACE2 binding cyclic peptide with an intramolecular disulfide bond, were evaluated in vitro, in preclinical model systems, and in a first-in-human translational ACE2 imaging of healthy volunteers and a SARS-CoV-2 recovered patient.

### Methods

All solvents and chemicals purchased from commercial sources were of analytical grade or better and were used without further purification. DX600, NODAGA-DX600, and DOTA-DX600 were custom synthesized by ChinaPeptides Co., Ltd (Shanghai, China) or CSBio (San Diego, Calif.). Sep-Pak Accell Plus QMA and Sep-Pak C18-Light cartridges were purchased from Waters (Ireland). Acrodisc 25 mm Syringe filter (0.22 μm) was purchased from Pall Corporation (USA). The product was analyzed by radio high-performance liquid chromatography (HPLC) (1200, Agilent, USA) equipped with a γ detector (Flow-count, Bioscan, Washington. D.C., USA), using a C18 column (Eclipse Plus C18, 4.5×250 mm, 5 μm, Agilent, USA). The product purity was also determined using Radio-TLC (AR 2000, Bioscan, USA) after radiolabeling. The PET/CT imaging studies of small animals were performed on the Mira® PET/CT of PINGSENG Healthcare Inc. (Shanghai, China), or microPET R4 rodent scanner (Siemens) and analyzed by ASIProVM. The Clinical PET/CT scans were obtained on a Biograph mCT Flow 64 scanner (Siemens, Erlangen, Germany) with unenhanced low-dose CT. 68Ga-HZ20 PET/MRI was performed on a hybrid 3.0T PET/MR scanner (uPMR790, UIH, Shanghai, China) in female volunteers.

Radiolabeling and Quality Control of Radiopeptides

⁶⁸Ga-HZ20: 195 μL 1.0 M NaOAc solution containing 40 μg (1.17×10-5 mmol) DOTA-DX600 was added into 3.0 mL of ⁶⁸GaCl₃ freshly eluted from ⁶⁸Ge-⁶⁸Ga generator (Isotope Technologies Garching, Germany) by 0.05 M of hydrochloric acid, with the radioactivity ranging from 370 to 1110 MBq. The final pH of the reaction was controlled at 4.2 and the mixture was heated at 95° C. for 15 min. After the reaction completed which was monitored by radio-TLC, the mixture was loaded onto an activated Sep-pak C18 cartridge. The cartridge was first washed with 5 mL of water to remove the free ⁶⁸Ga, and then eluted with pure ethanol to obtain the product of 68Ga-HZ20 as a 0.5 mL ethanol solution. The ethanol solution of the product was diluted with 10 mL 0.9% saline and filtered through a 0.22 μm filter (Merk, Darmstadt, Germany) to make the radiopharmaceutical used in this study. It contains 37-370 MBq of 68Ga-HZ20 in ethanol-water (ethanol <5%).

The solution was analyzed by radio-HPLC to assess the radiochemical purity. The HPLC was eluted with water-CH₃CN system (Phase A: 0.1% TFA H₂O; Phase B: 0.1% TFA CH₃CN) using gradient elution (0-5 min 20% B; 5-10 min 20%-80% B; 10-12 min 80% B; 12-15 min 80%-20% B) at a flow rate of 1.0 mL/min. The radiolabeling yield was 59.9±3.9% (non-decay corrected, n=10) and the radiochemical purity was over 95%. In vitro stability study of 68Ga-HZ20 in phosphate buffer saline was performed by adding 50 μL of 68Ga-HZ20 to 450 μL of phosphate buffer saline and incubating at 37° C. At 1 h, 2 h, and 4 h time points, 10 μL aliquot was analyzed by radio-HPLC to assess the radiochemical purity.

^{64/nat}Cu-HZ20: ⁶⁴Cu-labeled NODAGA-DX600 was prepared by dissolving 20-40 μg of peptide in 10-20 μL of trace-free water and 50 μL of 0.25 M ammonium acetate buffer (pH 6.0), and adding 37-74 MBq ⁶⁴CuCl₂ solution (1-2 μL) followed by a 5 min incubation at 95° C. After incubation, the final product was analyzed with analytical HPLC (Column: Kromasil 100-5-C18, 4.6×150 mm; flow rate: 1.0 mL/min; mobile phase: 0.1% TFA in water and CH₃CN, gradient: 0-10 min, 10%-50% CH₃CN, 11-14 min, 95% CH₃CN, 15 min, 10% CH₃CN), and reformulated with PBS/BSA (1.0% bovine serum albumin) solution for further experiments. If needed, purification with HLB cartridge (Phenomenex. Torrance, Calif.), For binding studies, one equivalent of natCu(SO₄)₂ was further added into reaction mixture, and the final solution was incubated for another 5 min to generate structurally identical ^{64/Nat}Cu-labeled HZ20 for saturation binding studies.

Surface Plasmon Resonance Assay

The binding affinity between DX600 (or DOTA-DX600) and ACE2 was determined by surface plasmon resonance (SPR) assay with Nicoya Open SPR system (Nicoya Lifesciences Inc., Ontario, Canada), in comparison to DX600. Briefly, recombinant human ACE2 protein (50 μg/mL; BP003061) was immobilized on the surface of the nanogold sensor chip, after the chip was activated by 1-Ethyl-3-(3-dimethylaminopropyl)carbodiimide/N-hydroxysuccinimide. Various dilutions of DX600 or DOTA-DX600 were added at a flow rate of 20 μL/min for 7 min and the SPR signal was collected. KD, ka, and kd were calculated using Trace Drawer Evaluation version 2.0 (Trace Software International, Saint-Romain, France).

Saturation Affinity of ^{64/Nat}Cu-HZ20 Binding to the hACE2

Saturation affinity studies were performed in HEK293-hACE2 cells and HEK293-WT using various concentrations of ^{64/Nat}Cu-HZ20. Triplicate samples containing 0.28×106 cells and 0.01-2000 nmol of ^{64/Nat}Cu-HZ20 in 0.25 mL cell culture medium were incubated at 37° C. for 1 h and 4° C. for 24 h, respectively. The cells were collected with glass microfiber filters, washed with 4×2 mL of ice-cold TBS (pH 7.4), and radio-assayed with a gamma counter. ^{64/Nat}Cu-HZ20 uptake (molecules per cell) in the HEK293-hACE2 cells was plotted versus ^{64/Nat}Cu-HZ20 concentration, and Kd values were estimated using a least-squares fitting routine (GraphPad Prism 8, San Diego, Calif.).

In Vitro Uptake of ⁶⁴Cu-HZ20 in hACE2-Positive Cells

hACE2-positive cells (HEK293-hACE2) and HEK293-WT cells were cultured in DMEM HG cell medium and pre-seeded in a 6-well plate the day before cell uptake experiments. Cells (0.1×106 in a total volume of 0.95 mL cell medium) in triplicate were mixed with approximately 26 kBq of ⁶⁴Cu-HZ20 in 0.05 mL PBS buffer (final concentration: 2.5 nM), and the cell mixtures were incubated at 37° C. for 1 h. Nonspecific uptake was determined by co-incubating with 10 μg of DX600. After incubation, the unbound radioactivity was collected, and then the cells were washed twice with cold PBS. Glycine buffer (pH 2.8) was used to incubate the cells for 5 min over an ice bath two times to remove the non-internalized radiotracer. After incubation, the cells were destroyed with 1 M NaOH and the residual was collected for measuring the internalized radiotracer. For kinetic uptake studies, the samples were incubated at 37° C. for 15, 30, 60, and 120 min. and the cells were measured with a gamma counter.

PET Imaging in Animals

⁶⁸Ga-HZ20: Four-week-old female athymic nude mice were purchased from SLAC Laboratory Animal Co. Ltd., China. 5×106 of HepG2 cells were inoculated into the right front shoulder region of the mice to build the xenograft tumor model with ACE2 expression. When the tumor volumes were estimated to be 450-800 mm3, the mice were used for the studies. Under isoflurane anesthesia, mice bearing HepG2 tumors (n=4 per group) were injected intravenously with 100 μL of 3.7 MBq ⁶⁸Ga-HZ20 for small animal PET. Scans were performed at 60, 120, and 180 min after administration. Unlabeled DX600 (50 mg/kg body weight) and 3.7 MBq ⁶⁸Ga-labeled tracers were co-injected into mice bearing HepG2 tumors (n=4 per group) for blocking study as a control group. Subsequently, animals were scanned at 60, 120, and 180 min post-injection. Regions of interest were defined over tumor, heart, liver, lung, kidney, and muscle and uptake computed relative to the injected activity.

⁶⁴Cu-HZ20: Female immunocompromised R2G2 mice from Jackson Laboratories (Bar Harbor, Me.) were used for subcutaneous inoculation of dual pseudotumors of hACE2 and control (non-expressing) HEK293T cells. The cells (3×10⁶) suspended in a 1:1 mixture of PBS and Matrigel were implanted on the shoulder area. Tumors were monitored and imaging commenced after reaching >200 mm3.

Dual xenografts-bearing mice or transgenic mice were injected with about 3.0 MBq (0.3 nmole) of ⁶⁴Cu-HZ20 via tail vein injection. At 1, 4, and 20 h post-injection, PET imaging was performed for 15 or 30 min on microPET R4 rodent scanner (Siemens) with the tumors centered in the field of view, and the animal under 2% isoflurane anesthesia. PET images were reconstructed by an iterative three-dimensional maximum a priori algorithm. The calibration factor of the PET scanner was measured with a mouse-sized phantom composed of a cylinder uniformly filled with an aqueous solution of 18F with a known activity concentration. ROI analysis of the acquired images was performed using ASIProVM (Siemens) and the observed percent injected activity of tissue (% IA/mL) was measured.

Following PET imaging, tumors were excised, embedded in optimal-cutting-temperature mounting medium (OCT, Sakura Finetek), and frozen on dry ice prior to cutting a series of 10 μm frozen sections. Digital autoradiography was performed on a phosphor imaging plate at −20° C. Phosphor imaging plates were read at a pixel resolution of 40 μm with a Cyclone system (Perkin Elmer). Following autoradiography imaging, the same section was stained with H&E and whole-mount bright-field images acquired in a similar manner.

The dual xenografts-bearing animals were randomly assigned into three cohorts and administrated approximately 3.0 MBq (0.3 nmole) of ⁶⁴Cu-HZ20. At 1, 4, and 20 h post-injection, the animals were sacrificed for tissue dissection. The organs of interest were collected, rinsed, blotted, weighed, and counted with a γ-counter (Perkin Elmer Wizard2). The total injected radioactivity per animal was determined from the measured radioactivity in an aliquot of the injectate. Data were expressed as a percent of injected activity per gram of tissue (% IA/g).

Immunohistochemistry: Harvested mouse tissues were formalin-fixed, paraffin-embedded, and sliced at 4 μm thickness. Normal human tissue blocks were acquired from a tissue bank. Sections were incubated with 3% H₂O₂ at RT for 10 min. Antigen was retrieved from the tissue in citric acid buffer (0.01 M, pH 6) with microwave heating for 2.5 min, followed by a 5 min cooling step. For normal human tissues, microwave heating was extended to several rounds of 3 min exposure. Tissues were blocked with goat antiserum for 30 min, stained with rabbit anti-ACE2 antibody (1:100 ab108252, Abcam) at 4° C. overnight. The washed sections were then stained with goat anti-rabbit secondary (PV-6001, Beijing Zhongshan Jinqiao Biologicals) for 30 min at RT. Subsequently, the sections were developed with 3, 3′ diaminobenzidine tetrahydrochloride (DAB), dehydrated in an alcohol gradient, and sealed in neutral mounting media, and scanned (Aperio Versa 200, Leica).

PET/CT Imaging in Healthy Subjects

Clinical ⁶⁸Ga-HZ20 Release: ⁶⁸Ga-HZ20 was prepared as above under GLP environment dispensing hot cell (NMC Ga-68, Tema Sinergie, S.p.A, Italy). The radiotracer met or exceeded release quality control and criteria (data not presented). Patients received tracer at a mean administered activity of 2.48 MBq/kg (±0.42 MBq/kg).

Subjects enrollment: The informed consent was obtained from the volunteers for publication. The inclusion criteria for healthy subjects included: 1) older than 18 years, 2) the ability to provide informed written consent, 3) a medical history without any significant comorbidities, including physical examination, electrocardiogram, hematology, and biochemistry. The exclusion criteria included: 1) liver and renal function dysfunction, 2) pregnancy or current lactation. Following the criteria, four groups of subjects (five males with age below 50, four males with age above 50, five females with age below 50, and six females with age above 50) were enrolled in this study, for a total of 20 subjects. The age ranged from 32 to 72 with a mean of 51.1±15.1 and the demographic data of all the volunteers were shown in Table S2.

PET/CT Examination procedures: No specific preparation was required for the subjects on the day of 68Ga-HZ20 PET scanning. A low-dose CT scan (120 kV, 35 mA, slice 0.6 mm, matrix 512×512) was performed before the ⁶⁸Ga-HZ20 injection. Then, a whole-body dynamic PET scan was performed immediately after the intravenous injection of ⁶⁸Ga-HZ20 in all subjects and continued for approximately 40 min (5 passes, round 10 min for each pass). All the subjects also underwent static whole-body PET/CT scan at 90 min and 180 min post-injection. Dynamic whole-body PET/CT scans were performed on a Biograph mCT Flow 64 scanner (Siemens, Erlangen, Germany) with the setting of 120 kV, 146 mAs, slice 3 mm, matrix 200×200, full width at half maximum (FWHM) 5 mm, filter: Gaussian, field of view (FOV) 256 (head), 576 (body). The patient bed was set to continuously move at a speed of 2 mm/s to cover the entire body of each subject (from the top of the skull to the middle of the femur). Static whole-body PET/CT scan used a speed of 1 mm/s at 90 min and 0.8 mm/s at 180 min. Three-dimensional iterative reconstruction was applied for image reconstruction, with CT-based attenuation and scatter correction through standard vendor-based reconstruction. The ⁶⁸Ga activities were decay corrected to the time of injection and normalized to the total activity administered.

Image Analysis: MultiModality Workplace (Siemens, Erlangen, Germany) was used for data processing. To analyze the biodistribution of ⁶⁸Ga-HZ20, regions of interest (ROIs) were manually drawn on the largest transverse section of major organs/tissues, while avoiding major blood vessels. The normal organs/tissues selected for VOI analysis included: the brain, parotid glands, nasal mucosa, oropharynx, nasopharynx wall, thyroid, cardiac muscle, left heart ventricle, lungs, liver, gallbladder, pancreas, spleen, kidneys, red marrow, bone, stomach, small intestine, upper and lower colon, rectus, breast, uterus, ovary, prostate, testis and muscle (quadriceps femoris). The maximum single-voxel standardized uptake values (SUVmax) were generated automatically from ROIs in MultiModality Workplace for analysis and comparison. SUVmax is defined as:

$$SUVmax = r \text{/} ( \frac{{a}^{′}}{w} )$$

where r is the maximum radioactivity activity concentration [kBq/mL] measured by the PET scanner within the defined ROI, a′ is the decay-corrected amount of injected radiolabeled ⁶⁸Ga-HZ20 [kBq], and w is the weight of the patient [g].

Statistics

Statistical analyses were performed with SAS software (V.9.4, SAS) and Prism (V8.0, GraphPad Software). The organ uptake data in the form of SUVmax were grouped by gender and by age. To compare distributions among samples, the parametric continuous variables were expressed as mean±SD. A mixed model was applied to compare the SUVmax of different groups for different time points, with study subjects as random effects. Independent sample t-tests were used to compare SUVmax values between different groups. A P-value less than 0.05 was considered to be significant.

### Results

DX600 is a high affinity ACE2 binding cyclic peptide with an intramolecular disulfide bond initially discovered from a phage display screen for ACE2 inhibition. It shows high selectivity on ACE2 over angiotensin-converting enzyme (ACE) and is stable to ACE2 catalyzed hydrolysis. The potency of the ligand was not predicted to be significantly diminished through modification with a radio-metal chelator, such as 1-(1,3-carboxypropyl)-1,4,7-triazacyclononane-4,7-diacetic acid (NODAGA) for stable ⁶⁴Cu labeling or 1,4,7,10-tetraazacyclododecane-1,4,7,10-tetraacetic acid (DOTA) for stable ⁶⁸Ga labeling, at the N-terminus of DX600 as it is distant from the key binding domain of CSPLRYYPWWKC (SEQ ID NO:2). Chelate conjugated-DX600 (FIG. 6A) was obtained in high purity (>98%) and characterized by mass spectrometry (FIGS. 13A and 13B), which are matching with the structure-based estimation. ⁶⁴Cu-HZ20 (FIG. 6B) was prepared quantitatively with a radiochemical purity of >97% within 30 min. ⁶⁸Ga-HZ20 (FIG. 6B) was produced in high radiochemical yield of 59.9±3.9% (non-decay corrected, n=10) with over 95% radiochemical purity. The specific activity of ⁶⁸Ga-HZ20 was determined to be 6.0×10⁴ GBq/mmol (n=3). ⁶⁴Cu/⁶⁸Ga-HZ20 with different decay half-lives (12.7 h vs 68 min) allowed us to conveniently carry out further studies.

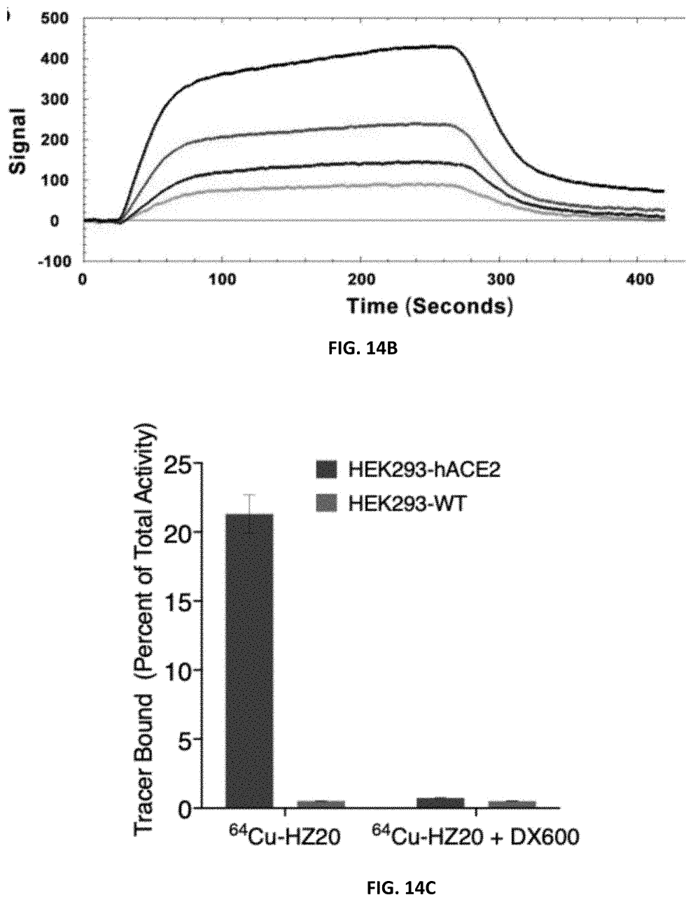

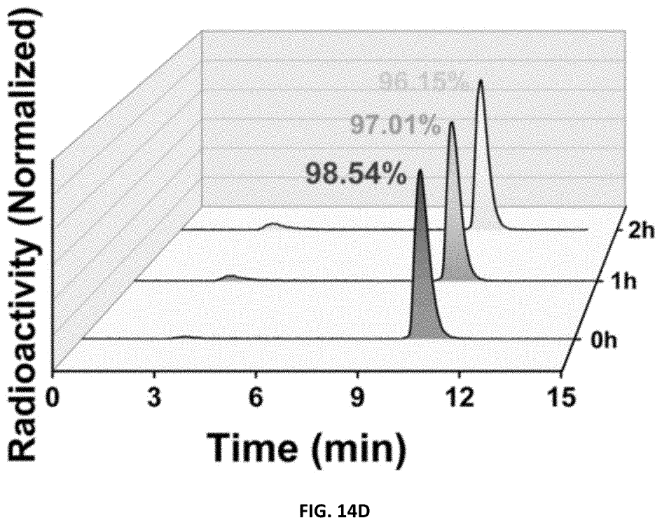

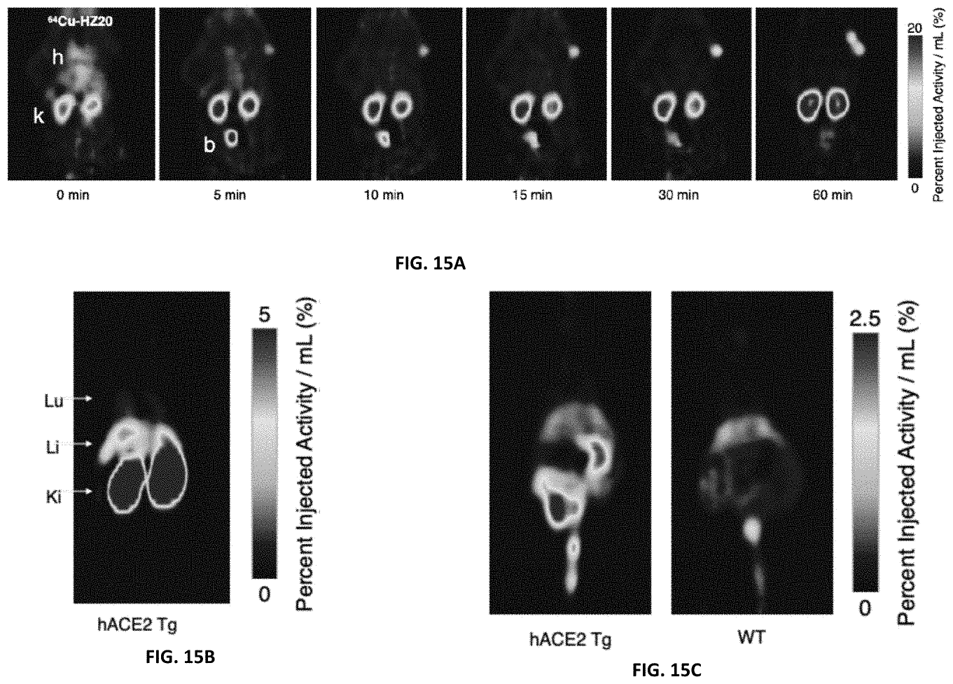

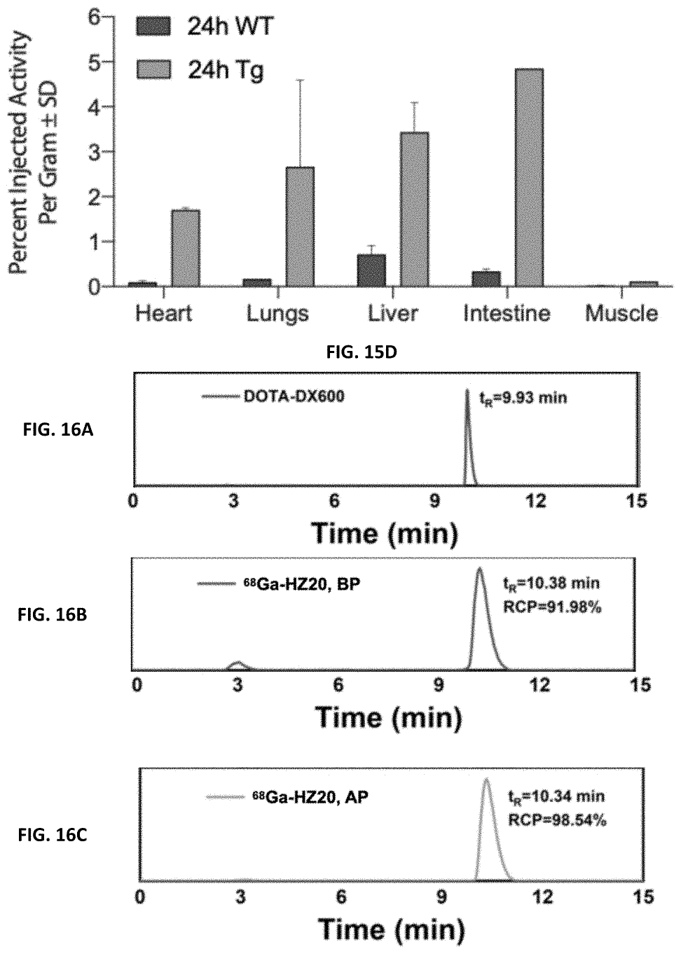

We initially tested the binding potency by using surface plasmon resonance (SPR). DX600 and HZ20 showed similar Kd of 98.7 and 100.0 nM, respectively (FIGS. 6D and 14B). The specificity of binding was assessed using a saturation binding assay with ^{64/Nat}Cu-HZ20 on ACE2-expressing and vehicle HEK293 cells at 4 and 37° C., respectively. ^{64/Nat}Cu-HZ20 displayed a higher binding potency toward ACE2 (66±1 nM) at 4° C. than the parental DX600 (FIG. 6C), a lower affinity (143±1 nM) at 37° C. with higher binding molecules (FIG. 14A), which indicated that the internalization of ⁶⁴Cu-HZ20 might exist at physiological conditions, and may be retained in target cells. Further in vitro cell uptake studies showed that the radiolabeled ACE2-binding peptide displayed a highly specific accumulation of ACE2-expressing cells in a time-dependent manner with significant retention (FIG. 14A), and 71±4% of total bound ⁶⁴Cu-HZ20 internalized (FIG. 6E).

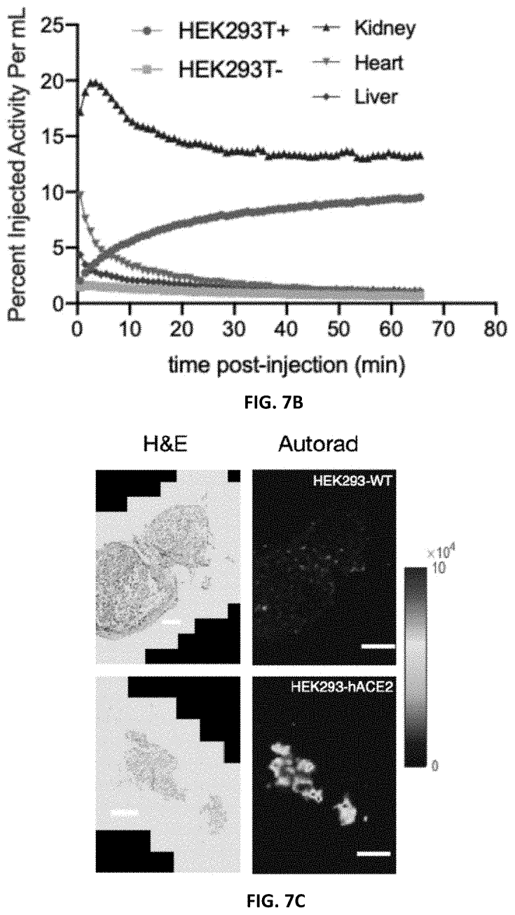

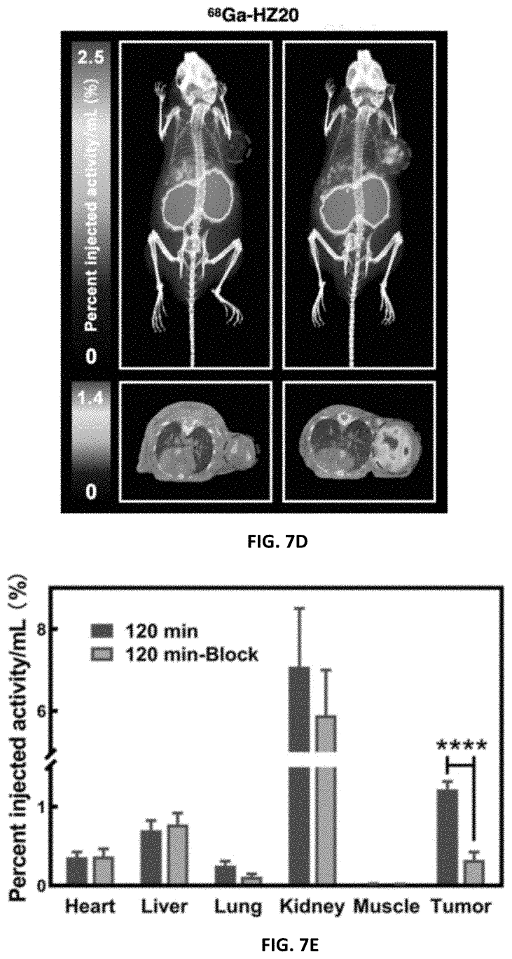

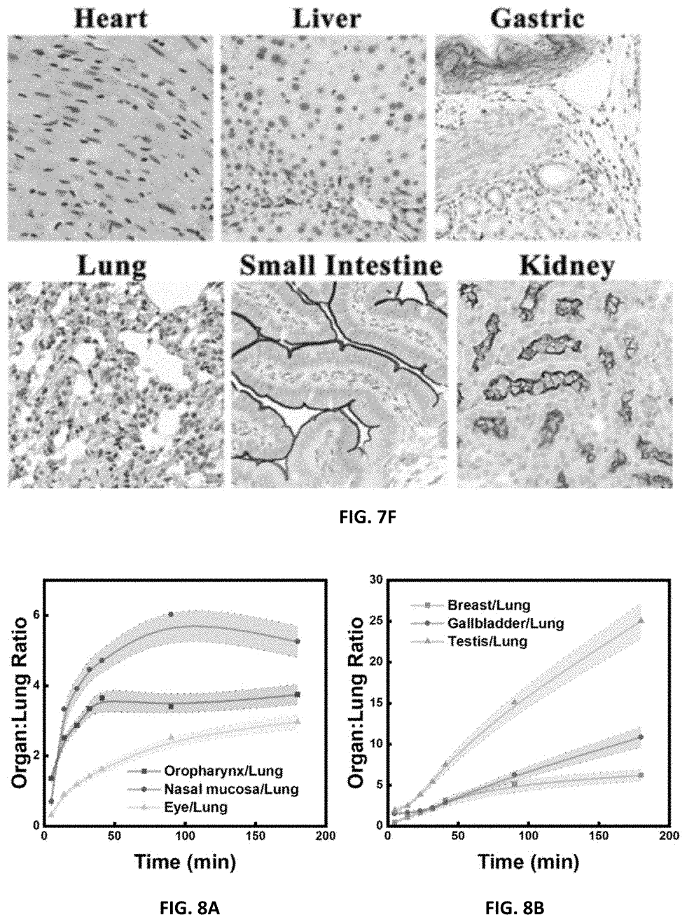

To evaluate the distribution, kinetics, and targeted uptake of HZ20 PET at human (h)-ACE2 sites in vivo, we evaluated two xenograft models of hACE2-expressing cell lines, an engineered HEK293T and the liver hepatocellular carcinoma HepG2, with high endogenous ACE2 expression. Both radioligands enabled clear visualization of ACE2-expressing xenografts following tail vein administration, and in vivo specificity was tested with cold blocking and ACE2-models and non-hACE2 expressing xenografts (FIG. 7A and FIGS. 14A, 14B, 14C, and 14D), which were also further verified by ex vivo autoradiography (FIG. 7C). The percent injected activity/mL (% IA/mL) of HZ20 accumulation in heart, liver, lung, kidney, muscle, and tumor at 1 h, 2 h, and 3 h were quantified by using ⁶⁸Ga-HZ20 PET imaging (FIGS. 18A and 18B). HepG2 tumor showed 0.56% IA/mL at 2 h, which was 96 times higher than that of muscle, and significantly blocked with co-injection of DX600. Similar to ⁶⁸Ga-HZ20, ⁶⁴Cu-HZ20 also showed the highest signal in the kidneys, high intensity in HEK293-hACE2 xenografts, and moderate in liver (FIGS. 7A and 7B). Kinetic analyses of this longer-lived tracer show persistent renal signal which coupled ACE2 immunohistochemistry suggest binding to the highly expressed murine ACE2 in this organ, for which radio-HZ20 ligands have greatly reduced affinity (FIGS. 14A and 14B). We further investigated ⁶⁴Cu-HZ20 using hACE2 transgenic mice. Compared to the distribution of ⁶⁴Cu-HZ20 in the wild-type strain, hACE2 transgenic mice showed significantly higher accumulation in the heart, lung, liver, and intestine, (FIGS. 3A, 3B, 3C, and 3D) which is consistent with the tissue profile of transgenic hACE2 distribution.

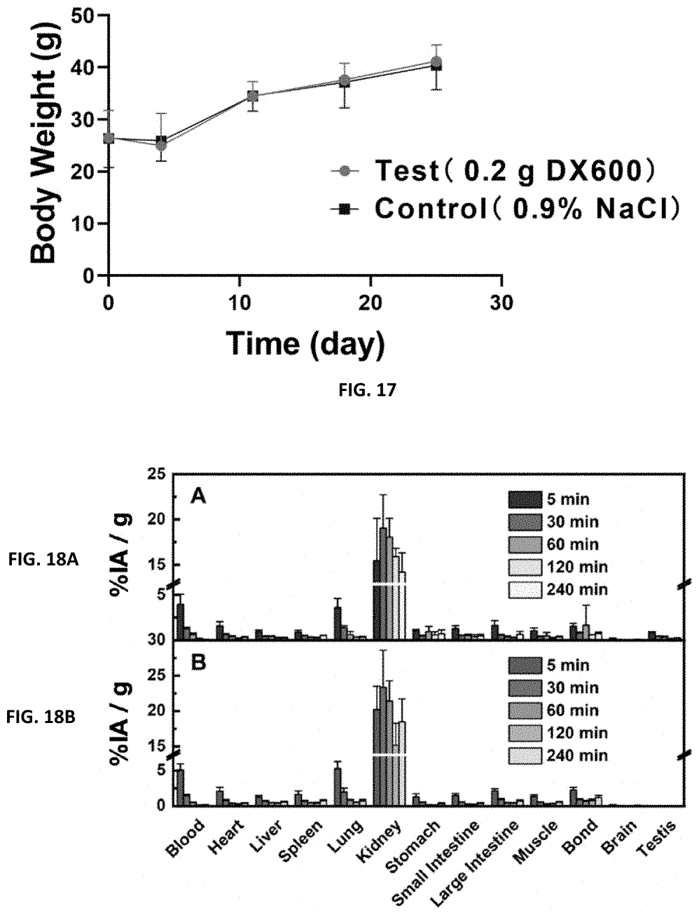

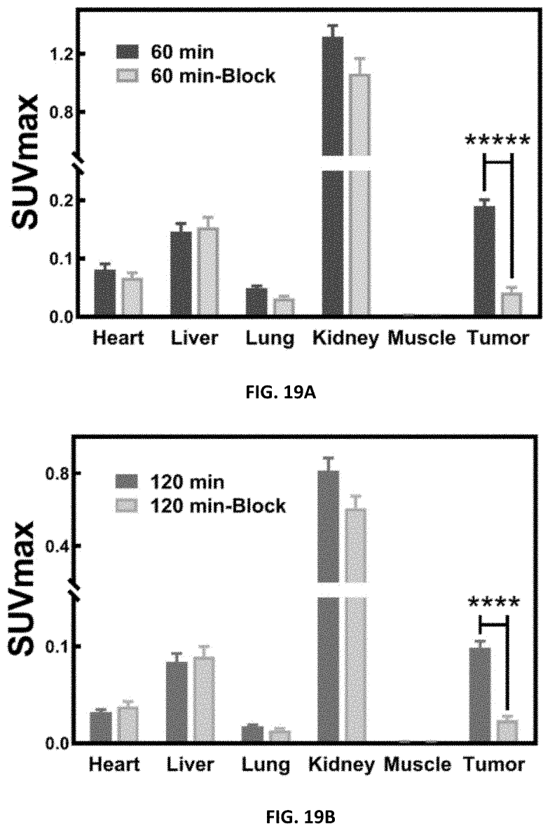

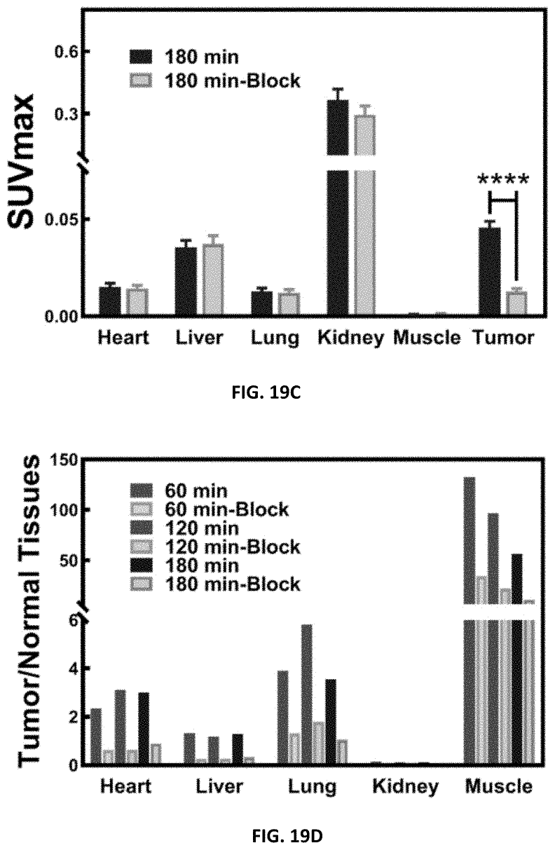

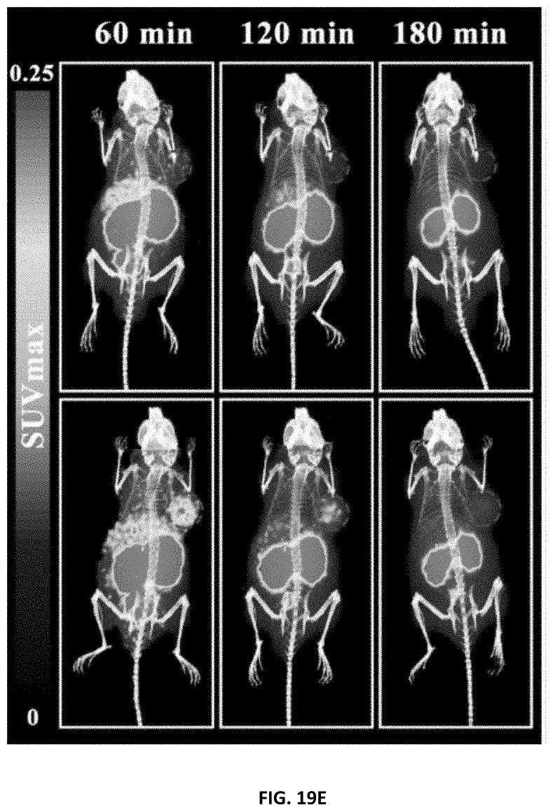

In vitro and in vivo experiments demonstrated the feasibility for ⁶⁴Cu/⁶⁸Ga-HZ20 to specifically monitor human ACE2 expression with high contrast ratios. The capability to perform later post-clearance imaging with cyclotron-produced ⁶⁴Cu (half-life 12.7 h) and logistical advantages are offset by the increased absorbed dose computed to the kidneys. The short-lived ⁶⁸Ga (half-life of 68 min) can be labeled into the DOTA chelator and is easily accessible through a network of widely available ⁶⁸Ge/⁶⁸Ga generators. In this study, we performed cGMP production of ⁶⁸Ga-HZ20 in the hospital, ⁶⁸Ga-HZ20 was proved to be stable within 2 h and passed all quality control standards for clinical trial. Preclinical dosimetry was performed to determine a lead ligand for first-in-human evaluation, which showed that the shorter half-life of Gallium-68 was more amenable for human study (FIGS. 16A, 16B, 16C, 17, 18A, and 18B). After an acute toxicity test proved ⁶⁸Ga-HZ20 to be well tolerated (FIG. 17), we initiated a clinical PET/CT imaging study in 10 volunteers.

Five males and five females were enrolled and underwent multiple whole-body PET/CT examinations to compute the mean organ absorbed dose per unit of administered radioactivity. The effective dose of ⁶⁸Ga-HZ20 to male and female were calculated as 0.017 mSv/MBq and 0.016 mSv/MBq, both of which are lower than the whole-body effective dose of 0.019 mSv/MBq as reported for the ICRP for 18F-Fluorodeoxyglucose (18F-FDG, the most commonly used PET radiopharmaceutical).

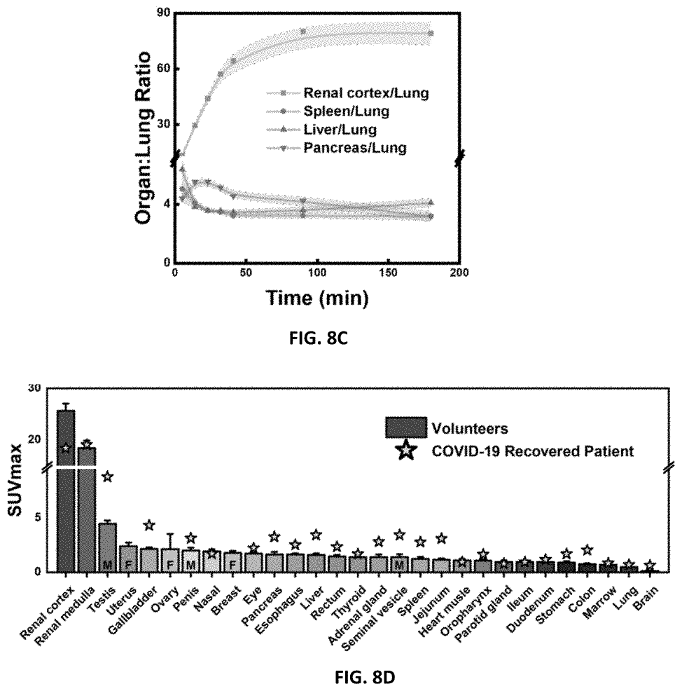

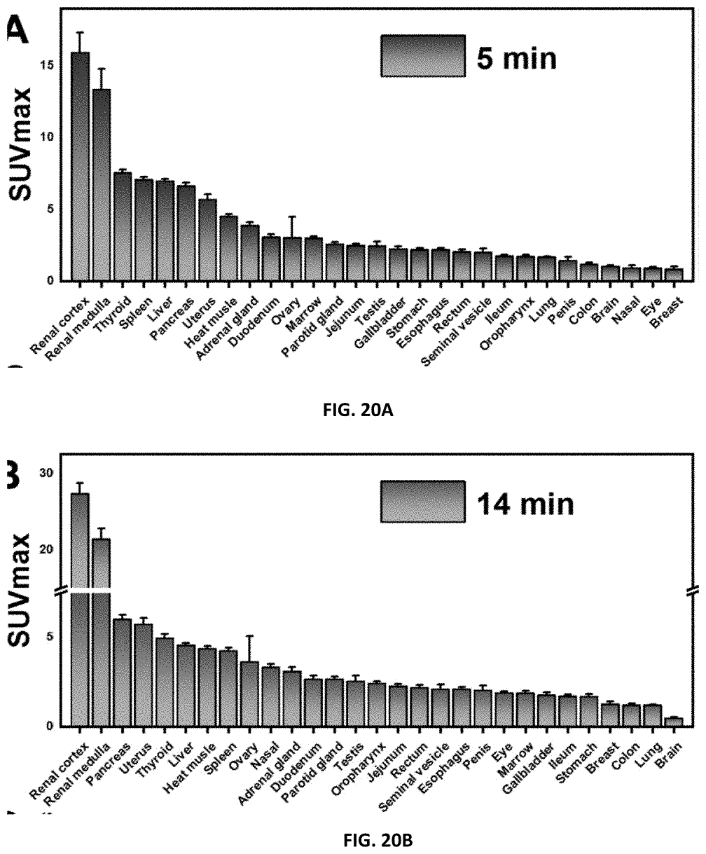

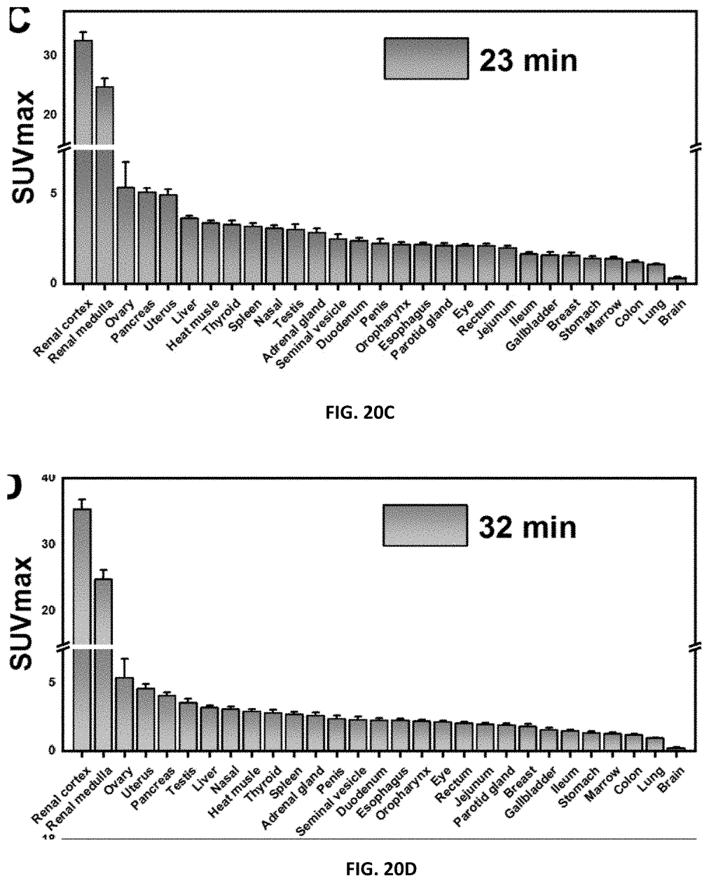

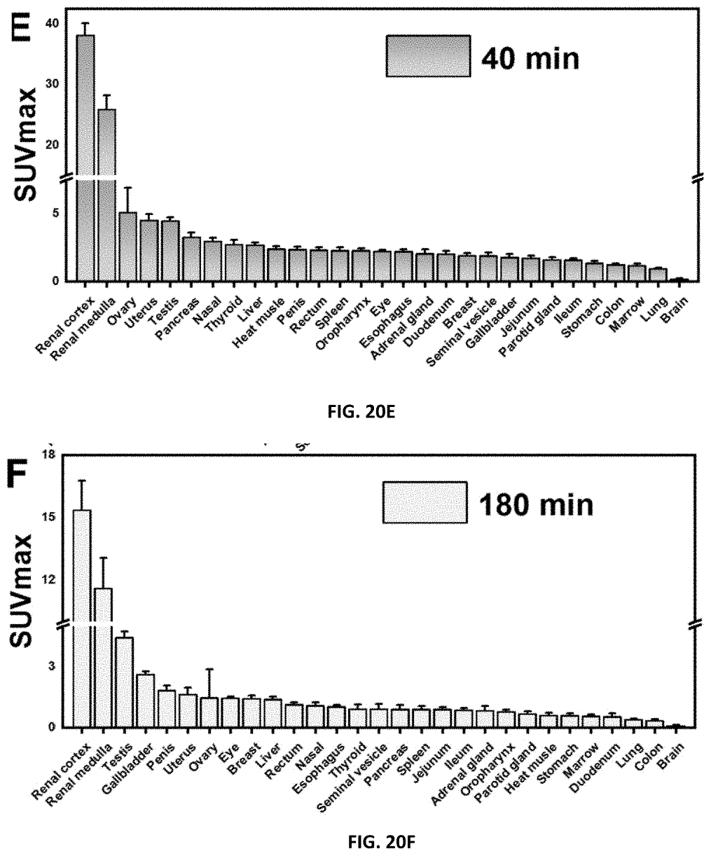

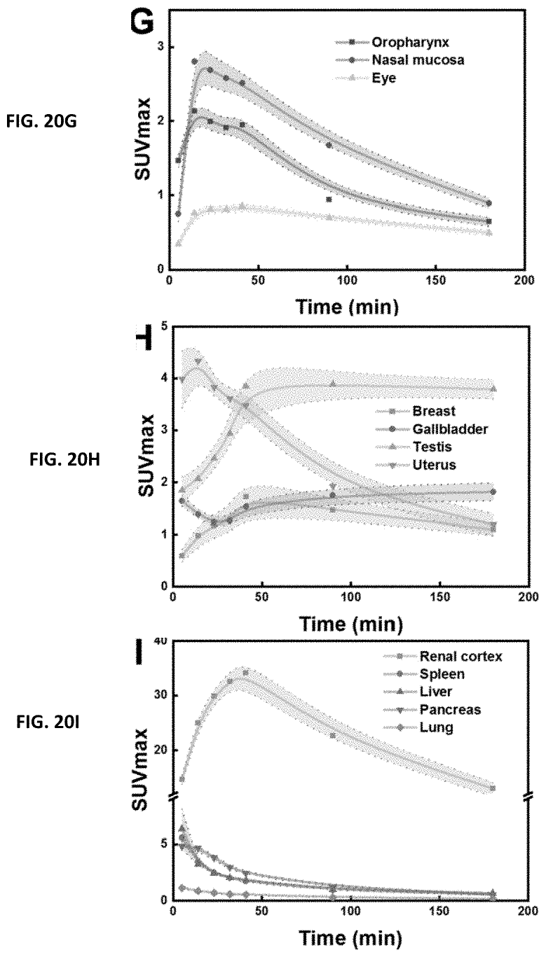

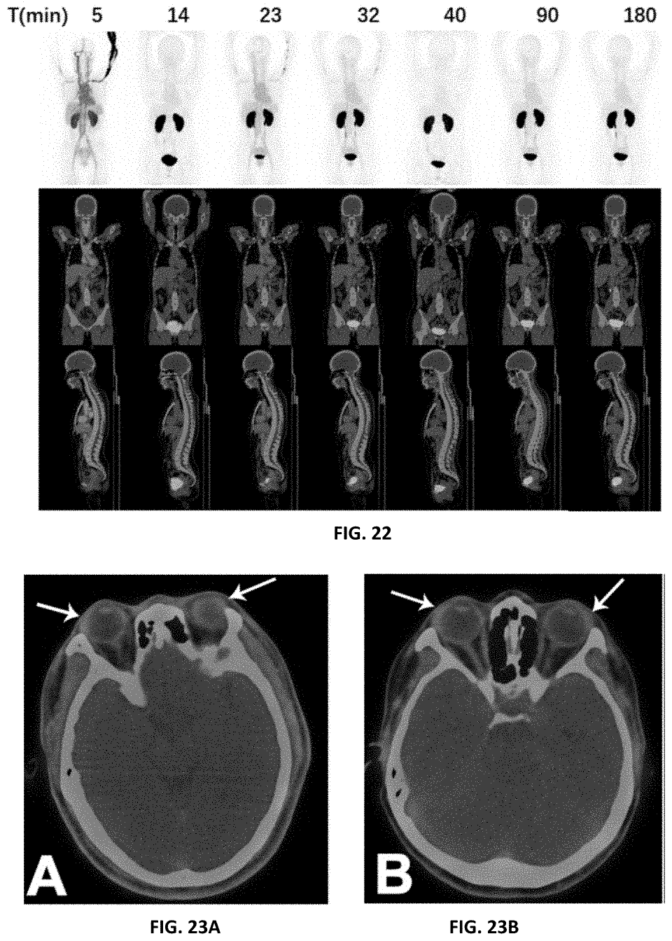

With the radiation safety on ⁶⁸Ga-HZ20 determined, we enrolled an additional 10 healthy volunteers and carried out a study in a total of 20 subjects with the aim to quantify the ACE2 distribution in different organs. 1.85-2.96 MBq/kg of ⁶⁸Ga-HZ20 was applied as the injection dose. PET data were acquired for all the subjects beginning immediately after administration of ⁶⁸Ga-HZ20, and seven scans were performed for all the subjects (5 min, 14 min, 23 min, 32 min, 41 min, 90 min, and 180 min). The images of a representative male volunteer are shown in FIG. 22. The standard metric for tracer accumulation is reported at each time point, which measures the bodyweight normalized maximum concentration in a voxel within the region of interest (the standardized uptake value maximum; SUVmax). Organs including the nasal mucosa, oropharynx, conjunctiva, heart muscle, breast, gallbladder, renal cortex, testis, seminal vesicle, corpus luteum, and digestive tract, showed both an absorption and elimination phase, indicating ACE2-specific uptake of ⁶⁸Ga-HZ20 (FIGS. 8A, 8B, 8C, 20A, 20B, 20C, 20D, 20E, 20F, 20G, 20H, and 20I). Rapid elimination from brain, thyroid, lung, liver, spleen, adrenal gland, pancreas, and uterus, suggest relatively low ACE2 expression levels, and the radioactivity gradually clears from the blood pool (FIGS. 8C, 20A, 20B, 20C, 20D, 20E, 20F, 20G, 20H, and 20I). Balancing the factors of ACE2-specific uptake and blood pool clearance we posit that static imaging at 90 min may optimally reflect the ACE2 distribution. A static whole-body PET/CT scan at 90 min was selected for comparative data across patients and cohorts, and the average organ SUVmax of ⁶⁸Ga-HZ20 is summarized (FIG. 8D). As expected for a peptide-based ligand, poor blood-brain barrier penetration is noted resulting in little accumulation of activity in brain. The greatest accumulation of signal was observed in kidney (SUVmax of 25.67±1.39 in renal cortex and 18.34±1.42 in renal medulla), which may be caused by the co-effects of ⁶⁸Ga-HZ20 excretion and high kidney ACE2 expression. The reproductive system showed a relatively high SUVmax, including uterus (2.41±0.34), ovary (2.12±1.42), breast (1.78±0.17) for females; and testis (4.46±0.30), penis (2.00±0.25) for males. Other organs with high SUVmax included nasal (1.91±0.18), conjunctiva (1.71±0.08), pancreas (1.63±0.24), esophagus (1.63±0.11), and liver (1.59±0.14). There is continuing interest in the gastrointestinal system as a viral entry site, and indeed moderate radioactivity uptake was detected with the gallbladder and rectum having high SUVmax (2.14±0.16 and 1.46±0.13). However, ⁶⁸Ga-HZ20 PET imaging showed only low SUVmax in the healthy volunteer lung, even though lung is the critical organ for SARS-CoV-2 infection, which is consistent with the data from Human Protein Atlas database.

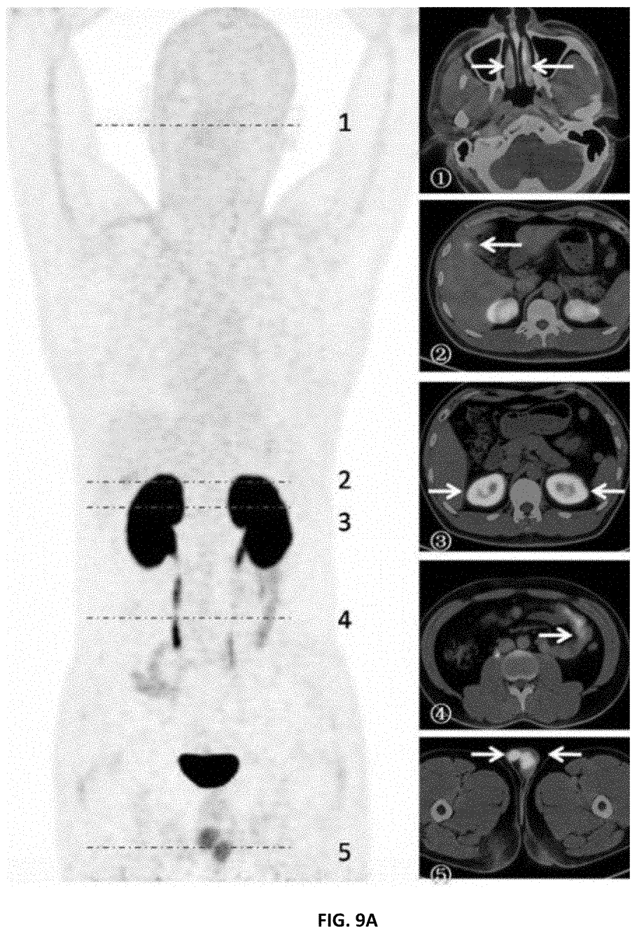

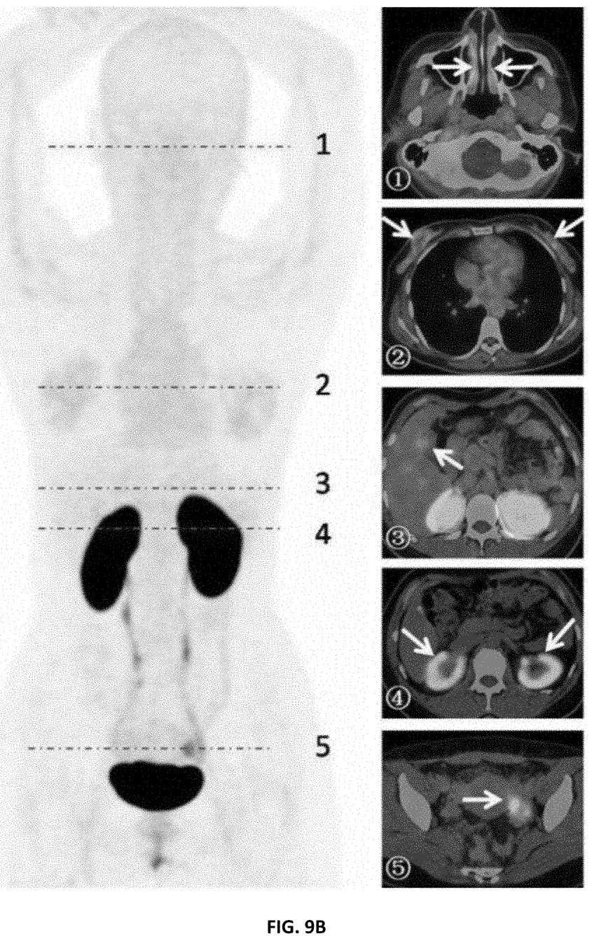

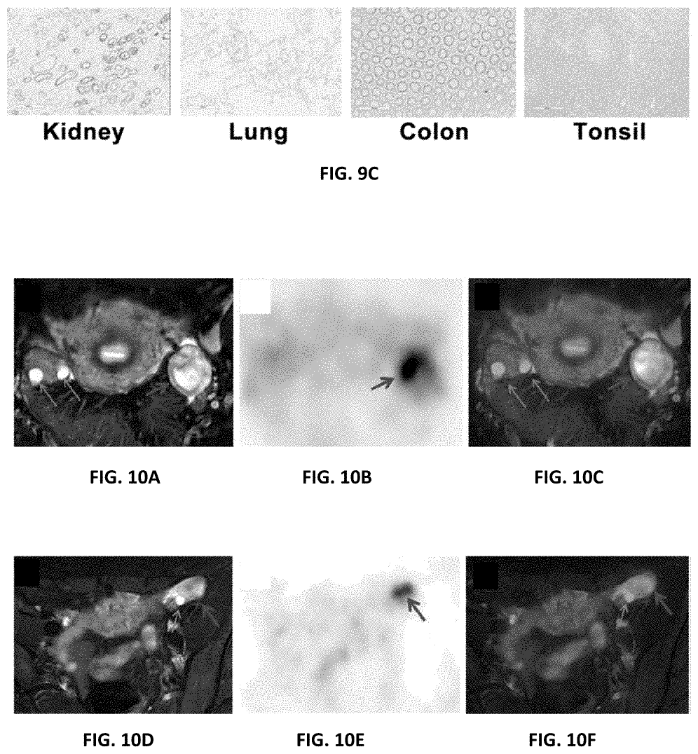

The representative whole-body PET of a 38-year-old male and 34-year-old female at 90 min post-injection and immunohistochemical analysis of human tissues are shown in FIGS. 9A and 9B. For the male, the SUVmax value showed the nasal mucosa (1.57), gallbladder (0.75), and small intestine (0.48) had moderate accumulation, and the renal cortex (23.88) and testis (5.57) showed high accumulation (FIG. 9A). Analysis in the female showed the nasal mucosa (2.54), breast (2.72), and gallbladder (2.76) had moderate accumulation, while the renal cortex (38.77) and the corpus luteum of left ovary (6.85) showed high accumulation (FIG. 9B). The high kidney uptake is in agreement with our preclinical and human immunohistochemical findings.

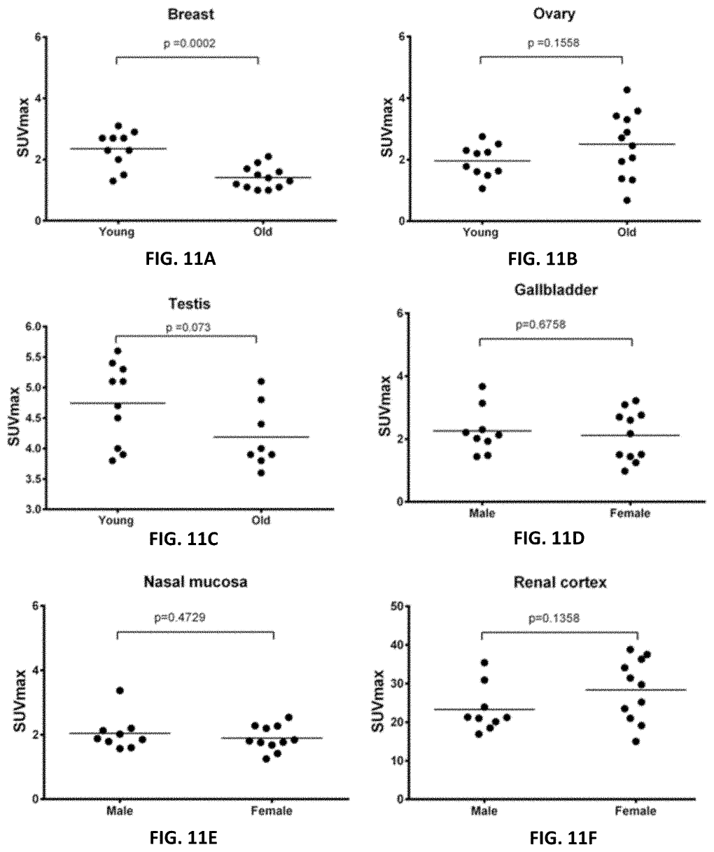

ACE2 distribution imaged by ⁶⁸Ga-HZ20 reflects sites implicated in the clinical manifestations of COVID-19 pathology could be clearly visualized with good contrast. The renal cortex, corpus luteum, and testis display high ⁶⁸Ga-HZ20 uptake (SUVmax>2.5). The breast, gallbladder, ovary, nasal mucosa, and esophagus showed median uptake for the young female subject (SUVmax 1.5-2.5), with relatively low uptake (SUVmax <1.5) in other organs. Additionally, the high ACE2 expression in conjunctiva as reported, was also visible on PET/CT (FIGS. 11A and 11B).

Interestingly, transient ACE2 uptake in the corpus luteum was observed compared to the immature follicles; and ⁶⁸Ga-HZ20 contrast was visualized in the ovaries of two young ovulating female volunteers (top row, SUVmax of 7.35 vs 1.95; bottom row, SUVmax of 6.85 vs 1.93, FIGS. 10A, 10B, 10C, 10D, and 10E). In order to investigate if identification of previously unidentified and transient ACE2 expression changes may be present, we conducted a delayed PET/MR (2 hours after ⁶⁸Ga-HZ20 injection) examination immediately following PET/CT imaging to reveal the precise structure of ovarian foci (FIGS. 10A, 10B, 10C, 10D, and 10E). The stroma of ovary showed slight uptake, and there was no uptake in immature follicles.

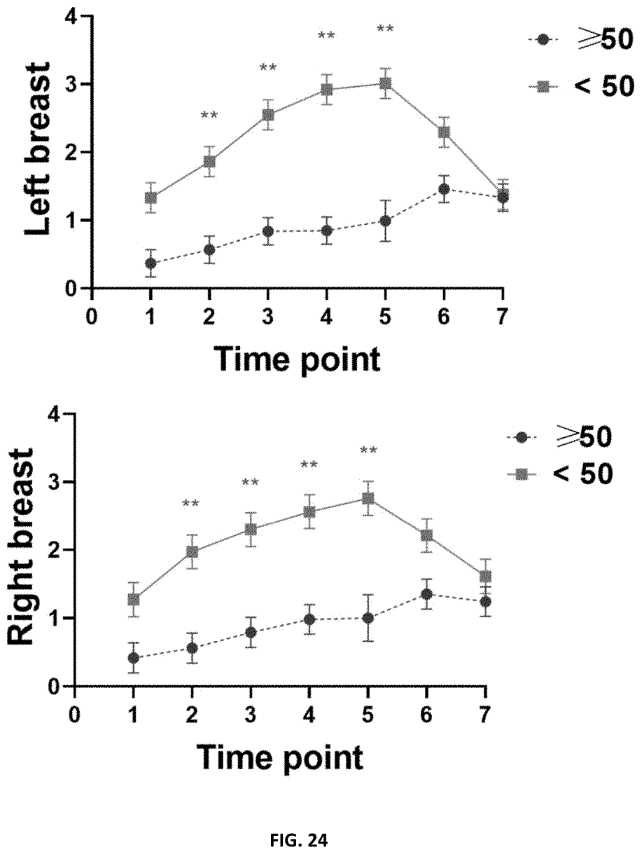

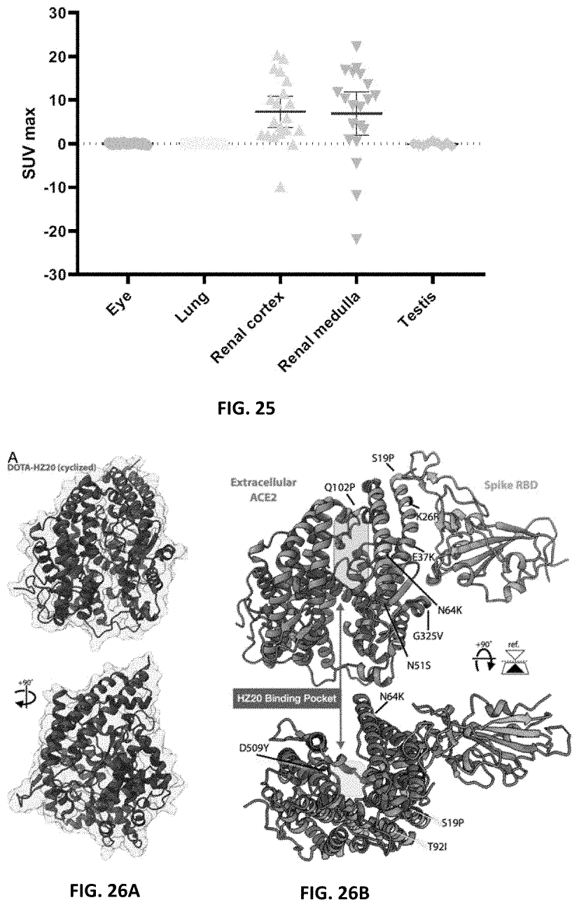

Clinical severity and mortality of COVID-19 have been more severe for men than women, in several studies and the aged population is particularly susceptible. To investigate the relationship of ACE2 across these populations we compared organ uptake by ⁶⁸Ga-HZ20 PET (FIGS. 11A, 11B, 110, 11D, 11E, 11F, and 24). A strong correlation was discovered between the ACE2 expression in the breast of female volunteers and with age (50-year as the cutoff), with the young group higher than the old group (2.21±0.25 vs 1.35±0.22, p=0.0002, at 90 min, FIGS. 11A, 11B, 11C, 11D, 11E, and 11F). At this time, we continue to increase the sample size to confirm these and other observations, a limitation of this first-in-man study.

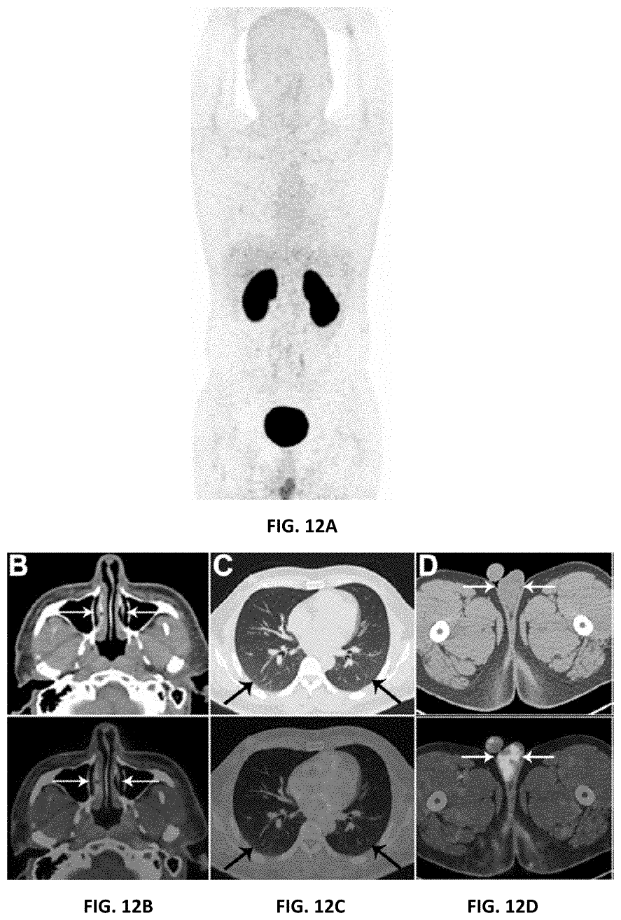

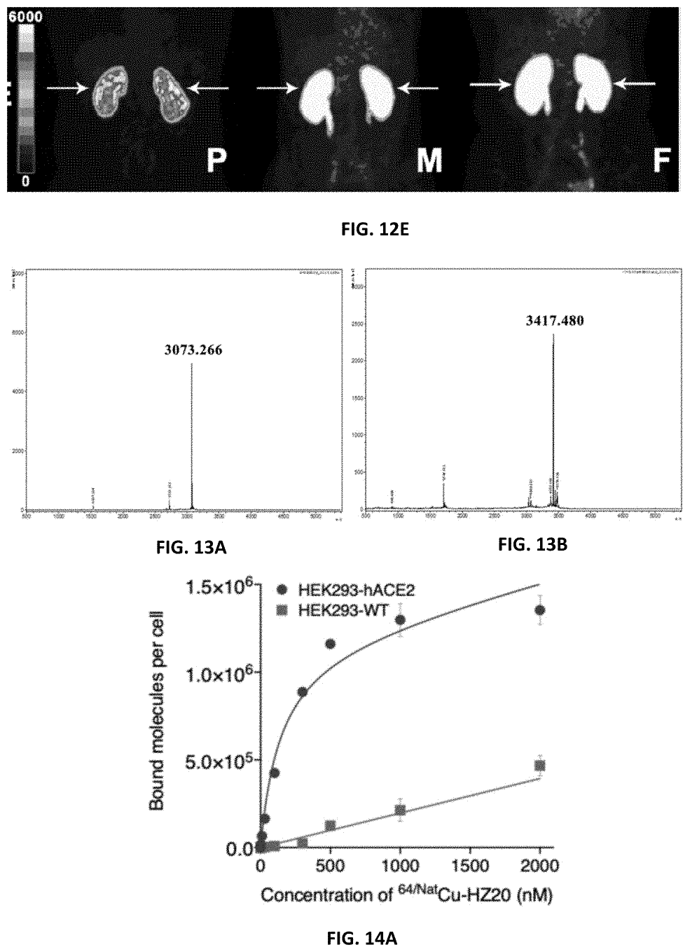

We next tested ACE2 PET in a 38-year-old male infected with SARS-CoV-2 at 7 months post-infection. Images at 90 min post-injection are shown in FIGS. 12A, 12B, 12C, 12D, and 12E, and all organs with high radioactivity uptake were visualized clearly with SUVmax of each organ determined (FIG. 8D). Evaluation of a larger number of recovered patients is required to draw robust conclusions, here the gallbladder, testis and many of the normal organs showed increased uptake versus the healthy volunteer pool. Conversely, renal cortex accumulation of the tracer was substantially lower (SUVmax: 18.40 vs 25.67±1.39). Acute kidney injury has been reported in about 9% of patients hospitalized with COVID-19, and these results were unexpected. While speculative, the differences in this patient suggest an organ-specific response, at the molecular expression level, resulting from infection even at these extended times post-infection. Extension and verification of these results in a wider population of COVID-19 patients will provide additional insight.

### Discussion

The ACE2 expression level and organ-specific distribution may potentially reflect the susceptibility, severity, and prognosis of SARS-CoV-2 infection. A non-invasive imaging tool to explore expression characteristics of ACE2 in vivo may assist in understanding the pathogenesis of SARS-CoV-2 infection with the potential to assist in development of mitigation and treatment planning. A high affinity human ACE2 specific peptide, DX600, was selected and modified as a PET imaging agent for translational evaluation. Radiometal-chelator conjugation did not affect binding nanomolar potency or specificity of the ligand, which was rapidly internalized. Preclinical investigation of ⁶⁸Ga/⁶⁴Cu-HZ20 demonstrated ideal pharmacokinetic properties in models of human ACE2 expression, with rapid blood clearance, low background organ uptake, and predominant renal clearance. High contrast of ACE2 pseudotumors was achieved within 2 hours and within genetically engineered models.

After safety and dosimetric evaluation, ⁶⁸Ga-HZ20 was applied for a first-in-human translational study with 20 healthy volunteers of different ages and both sexes. To fully characterize this molecular imaging tool, dynamic scans were performed for each volunteer. The results showed highly consistent pharmacokinetics among the 20 volunteers, with high ACE2 expressing organs (such as kidney, gallbladder, testis for male) reflected in both high SUVmax and signal retention within the 3 hours tested. The plateau of signal in expressing tissues and clearance from the blood pool affords convenient 90 min post-administration static images useful to profile ACE2 expression. Imaging results were mostly consistent with the HPA database (http://www.proteinatlas.org) and previous immunohistochemical profiling of ACE2, including microvilli of the intestinal tract and renal proximal tubules, gallbladder epithelium, testicular Sertoli cells and Leydig cells. Intriguingly, we report a differential finding between these imaging results and histological HPA data in the ACE2 expression in the female breast. We observed that the ACE2 expression level in breast is age-dependent, with medium for the young group and low for the old group (P=0.0002, FIGS. 11A, 11B, 110, 11D, 11E, and 11F), while HPA reported no ACE2 expression.

PET imaging of ACE2 non-invasively can provide real-time and global receptor distribution quantitatively using the SUVmax value of the uptake of ⁶⁸Ga-HZ20 correlating directly with ACE2 expression level, which offers novel information relevant for infection by SARS-CoV-2, and pathology of COVID-19. Recent reports have indicated that partial loss of the sense of smell or even total anosmia are early symptoms of SARS-CoV-2 infection. It was suggested that the virus may exploit goblet and ciliated cells in the nasal epithelia as entry portals, a plausible primary infection site in many patients. Along the respiratory tract, the nasal mucosa showed higher ⁶⁸Ga-HZ20 uptake than oropharynx, and the lung showed the lowest uptake. It should be noted that the lower density of the lung tissue likely biases measured uptake values to underestimate tracer concentration. These observations are consistent with a recent study using high-sensitivity RNA in situ mappings that have shown ACE2 expression is highest in the nose with decreasing expression throughout the lower respiratory tract, paralleled by a striking gradient of SARS-CoV-2 infection in proximal (high) versus distal (low) pulmonary epithelial cultures. This gradient could be clearly visualized in the same volunteer from the PET image.

Surprisingly, ⁶⁸Ga-HZ20 uptake in the lung and heart was very low. Data from the Human Protein Atlas and others have shown ACE2 receptor expression variability in patients bronchial and lung tissues with indications that underlying conditions inducing inflammation may have a role in expression levels. An interesting finding from these comparisons across patients in the imaged cohort is that there are negligible differences between young and geriatric males or females. These data potentially underline the equivalent risk of infection across all adults, and that further evaluation can be accomplished with the tracer noninvasively.

Studies have found that 70% of patients infected with SARS-CoV accompanied by diarrhea. A recent case report described that ACE2 is highly expressed in stratified epithelial cells and absorptive intestinal cells in the upper esophagus, ileum, and colon. These findings may support the possibility of fecal-oral transmission route, and also consistent with the uptake of ⁶⁸Ga-HZ20 in the oropharynx, intestine and rectum observed in our imaging results.

Several of our results highlight expression profiles in reproductive organs. Recent studies have reported testicular damage caused by SARS-Cov-2 infection, and investigation by PET on the reproductive function of recovered male patients, especially youth is warranted. Besides ACE2 expression observed in the breast of young females, ⁶⁸Ga-HZ20 uptake was observed in ovaries (without a difference between young and old women). We also found high ⁶⁸Ga-HZ20 uptake in corpus luteum in two young women, but not in immature follicles. ACE2 expression in antral follicles, mature luteal cells, the theca and stromal cell layer in ewes has been previously described, but to our knowledge this is the first observation of ACE2 in human corpus luteum with the direct comparison around surrounding organs. The ACE2 specific inhibition of the cyclic DX600 peptide has been established, however uptake at these unexpected sites may suggest non-specific interactions which can be definitively determined with future biopsy confirmation.

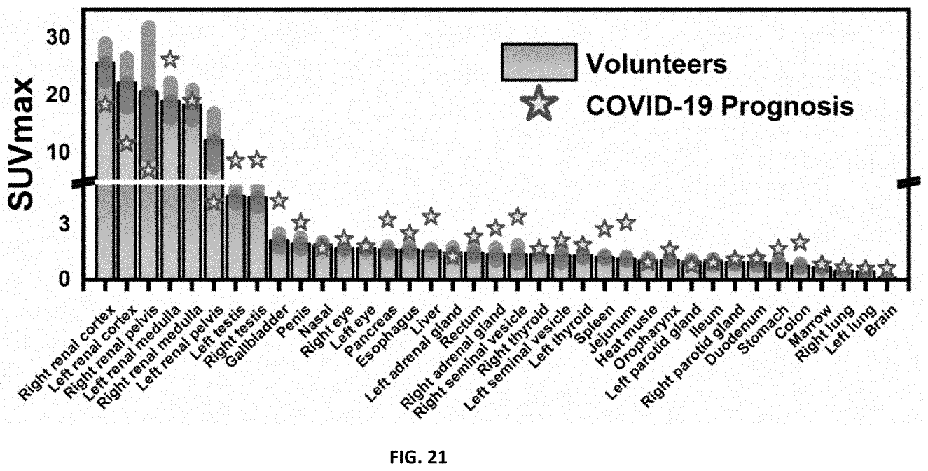

Acute kidney injury has also been commonly found in patients infected with SARS-CoV-2, with likely long-term impacts on overall health. In addition to the host's immune response, these acute sequelae of fighting the infection may also come from the direct attack of the virus on the target cells expressing ACE2. RNA-Seq studies have shown that ACE2 is abundantly expressed in various renal proximal tubule cell subtypes; consistent with our observations. Limited by qualified resources for carrying out studies in patients during the SARS-CoV-2 infection, we were only able to image one recovered volunteer in which we report dramatic changes in observed ACE2 expression (FIGS. 8D and 21). We stress that conclusions cannot be drawn across healthy volunteers and the recovered patient. However, these data strongly motivate further investigations in a larger recovered patient pool.

In summary, we have developed a non-invasive imaging method using ⁶⁸Ga/⁶⁴Cu-HZ20 PET to interrogate the global expression profile of ACE2 in humans, the key receptor for SARS-CoVs to infect human cells. To our knowledge, this is the first study to quantitatively profile ACE2 expression by PET imaging, and this approach demonstrates the capacity to measure organ express profiles at baseline and following COVID-19. The results from 20 healthy volunteers were consistent with pathological reports of receptor distribution, while preliminary imaging data from a SARS-CoV-2 infection recovered man may suggest ACE2 level changes. These preliminary results require further investigation in a wider patient population. With the capacity of quantitatively detecting stable and transient ACE2 expression non-invasively, this quantitative imaging method may have utility to evaluate differences across patients for SARS-CoV-2 infectivity; COVID-19 symptom severity and duration; and for evaluation of physiological effects from other emerging and novel coronaviruses.

### Example 3: Characterization of ACE2 Variants Binding by Imaging Agent

To quantify ACE2 levels across cohorts and in response to infection we will evaluate variants of the ACE2 protein. This is relevant because ACE2 genetic polymorphisms and truncated forms of the protein have been detected, shown to alter susceptibility, and modulate response to the virus. Validation of the disclosed imaging agent with ACE2 variants deepens our understanding and interpretation of [⁶⁸Ga]HZ20-PET radiological data. Here, we present modeling data for peptide-protein binding in the context of high-frequency variants. Two main types of variants have been described: a truncated (short- or delta-) ∂ACE2 and polymorphic mutations (below). ACE2 expression is controlled by multiple promoter elements, with lung and nasal epithelial regulated by interferon, STAT1, STAT3, and IRF1-binding sites. Activation of IFN-responsive genes is an important antiviral defense pathway, and both interferon and viral exposure have been reported to increase ACE2 expression in the human airway. Directly relevant here is that ∂ACE2 is specifically induced (over full-length ACE2) in response to IFN or viral exposure. Enzymatically nonfunctional, ∂ACE2 nor truncated MIRb-ACE2 product bind SARS-CoV-2 spike protein. Both are cell surface-expressed, retain the inhibitor region (FIGS. 26A and 26B), and the capacity of DX600-based [⁶⁸Ga]-HZ20, has not been determined.

Mutations are a core of the viral:host defense paradigm and ACE2 mutations (in particular the N90-linked glycan) render humans resistant to other animal-pathogenic coronaviruses. Binding of the HZ20 ligand to mutants is highly relevant as altered affinity of newly-detected ACE2 polymorphic variants for the SARS-CoV-2 spike protein has been established, impacting both susceptibility to infection and disease severity. Nearly 300 single amino acid substitutions, detected from human ACE2 population variation data sets, were identified, with many in the extracellular domain of ACE2 that alter the binding of the functional virus with implications for infectivity. As for the truncated ACE2 variants, many of the mutations may affect HZ20 binding (FIGS. 26A and 26B), and are conformationally adjacent to the peptide-protein docking structure we have determined using a hierarchical algorithm for flexible interactions. We will evaluate the binding capability of the [⁶⁸Ga]-HZ20 imaging agent to high-frequency amino acid substituted-ACE2 and truncated ACE2 variants revealed in patients. This will further establish the remit of the molecular imaging approach to report the expression of this key protein.

Computational Approaches for Enhanced DAR

To evaluate ligand binding with target ACE2 (and variants) on tissue we will perform digital autoradiography (DAR). This widely used approach in academia and industry for pharmaceutical development directly measures distribution at the tissue scale of radiolabeled compounds. Unlike optical methods, DAR exhibits a wide and linear response range, and exceptional sensitivity (fM). Limitations in resolution and background noise can result in poor correlation, even errors, between radiotracer distribution, anatomical structure, and molecular expression profiles. This is a challenging problem to overcome, as unlike conventional optical systems used for microscopy, the resolution of DAR is not fixed by wavelength or optics, but instead radioisotope decay, phosphor, and digitizer properties. Early efforts to overcome noise and blur-related artifacts included regularized iteration after noise filtration and modeling of noise features which were non-ideal and have not been widely adopted, in part, because of noise amplification effects.

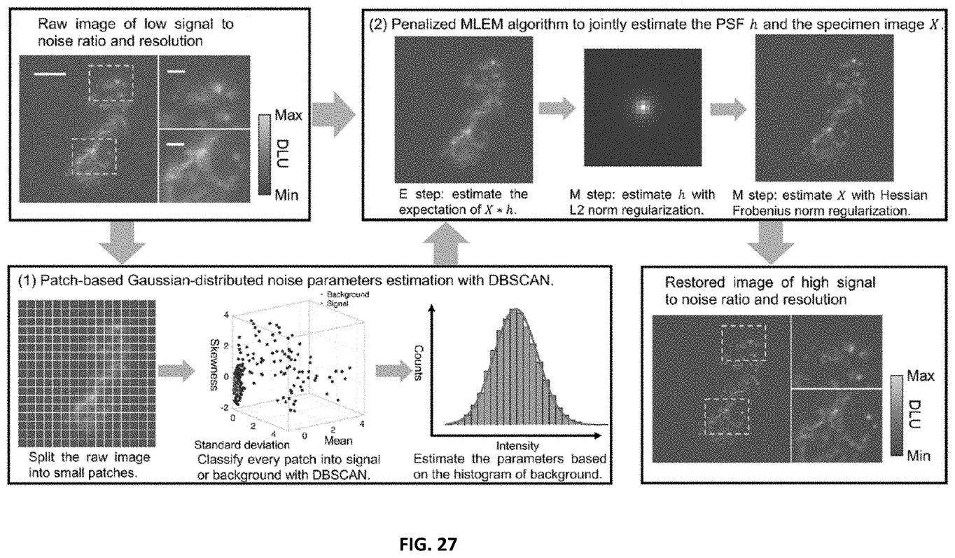

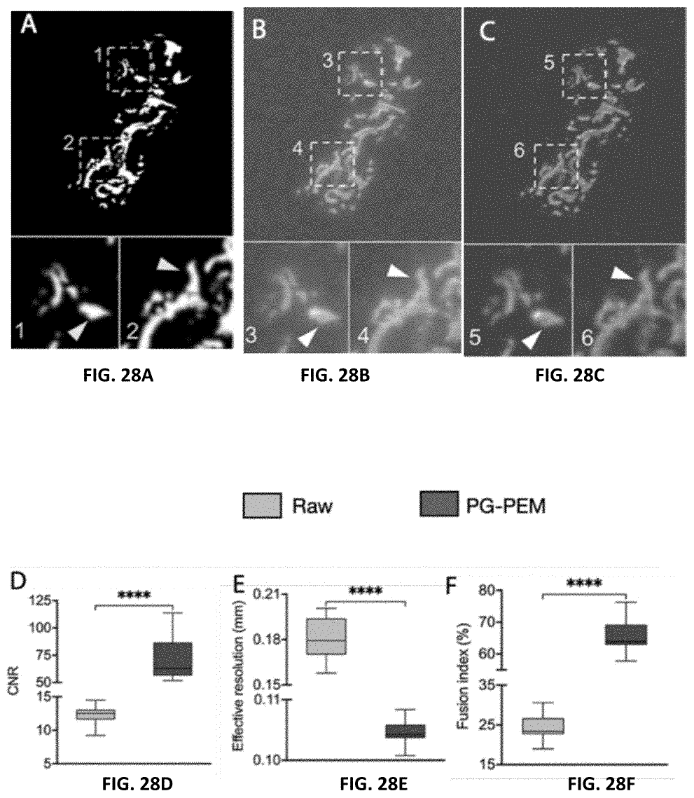

We have developed and implemented an approach to improve DAR resolution and quality while retaining quantitative accuracy that accounts for noise sources in the image formation process and an iterative reconstruction recovery process. The Penalized Maximum-Likelihood Expectation-Maximization algorithm (PG-PEM) implements a patch-based estimation DBSCAN algorithm to separate signal and background regions and applies regularization functions with biologically realistic features to jointly estimate the image point spread function. Importantly, the method is a so-called blind iterative approach meaning that a priori calibration (which is generally not feasible for stochastic decay properties of radioisotopes in the context of tissue-section samples). A visual depiction of the simultaneous computational processes of the algorithm is shown in FIG. 27. Using pre- and clinical samples, we will demonstrate that this approach improves resolution, decreases both spurious and background noise; and importantly enables improved registration with histopathological features. A representative example of a patient bone biopsy is shown in FIGS. 28A, 28B, 28C, 28D, 28E, and 28F with low activity Radium-223 uptake demonstrating the capacity of this technique to both improve quality and better align radiotracer distribution with anatomical features. We will leverage these approaches for quantitative ligand-binding DAR on tissue specimens from a COVID-19 Biospecimen tissue bank

Recombinant Variants and Testing: A large number of missense mutations have been discovered for ACE2, with a smaller number of truncated isoforms. We will focus on changes in the extracellular ligand-accessible domain and will choose 22 leads to test for HZ20 binding. These include: A) the 10 most common mutations from cancer patients derived from the Catalogue of Somatic Mutations in Cancer (COSMIC v91; (73)) at R115Q, R115W, R219H, D368N, E375D, F400L, D609N, R671Q, R708W, N720D, because cancer patients are more susceptible to adverse outcomes with COVID-19 (74-77). B) 10 high-frequency spike-affinity affecting mutations (S19P, K26R, T27A, K31R, N331, H34R, E37K, N51S, N64K, T921) due to changes induced here for SARS-CoV-2 infectivity. And C) the truncated ∂ACE2 and short-ACE2 as they have been detected in patients under viral-induced contexts

Extracellular hACE2 (1-615, NP_001358344) with the substitutions denoted above will be synthesized with a c-terminal GFP-tag (IDT) and cloned into a CMV-promoter driven mammalian expression vector. Sequence verified plasmids will be transfected into 293T cells and verified by microscopy (Nikon Ti2E), as well as western blot. Quantitative binding assays (with and without increasing blocking cold DX600) will be performed. Briefly 1×10⁵ cells in 1 mL DMEM mixed with 25 kBq of [⁶⁸Ga]-HZ20 (2.5 nM) in 50 μL PBS. Triplicate washing with 4° C. PBS and exposure to glycine (pH 2.8) and NaOH (pH 10) for separation of surface and internalized ligand for gamma (g)-counting. Kinetic studies will be performed at 0.25, 0.5, 1, and 2 h; Bmax and Scatchard analyses will be compared to full-length hACE2 293T.

### Example 4: [⁶⁸Ga]-HZ20 Pet for Patients Displaying Mild, Severe and Long-Covid-19

We will first conduct imaging in patients that are COVID-19 naïve to confirm our data from initial in-patient scanning. We will evaluate patients who had tested positive for infection during the pandemic with either mild or severe acute conditions. For a subset that had persistent symptoms, which have the potential to persist to the initiation of imaging, we will conduct repeat annual PET studies to monitor PET signal changes. This study will test the hypotheses that [⁶⁸Ga]-HZ20 signals correlate with disease severity; and patients who do not return to baseline health (long-COVID-19) have higher and possibly sustained ACE2 PET by repeat imaging.

We will evaluate a total of 87 subjects, both men and women (>18 yrs) for signal by [⁶⁸Ga]-HZ20 PET in yrs. An initial cohort of healthy COVID-19-naïve subjects (n=29) will be scanned at the outset to extend and establish baseline SUVmax values and to test for genetic/population effects between Chinese and American subjects.

At the time of imaging, we will record baseline medications, health status, and demographic information, as well as the patient's age and sex. All patients will have a 20 G antecubital IV placed for injection of a radiotracer. A 90 min uptake phase post-administration of 3 MBq/kg [⁶⁸Ga]-HZ20 per FDA eIND specifications will be followed by PET/MR. The Biograph mMR can simultaneously acquire radioligand and high-resolution soft-tissue detail. The initial 10 patients will have blood drawn at 5, 15, and 60 min, placed at 4° C. for serum purification and metabolite analysis.

An additional 58 patients will have had laboratory confirmed COVID-19. Patients will be selected that have had mild (self-reported asymptomatic, mild fever or dyspnea, no ventilation assistance) and severe COVID-19 (acute respiratory distress syndrome (ARDS) by bilateral infiltrates, severe hypoxemia, and lung edema, and/or hospitalization >72 h). Long-COVID-19 can manifest from either mild or severe cases, with reported symptoms with enrollment for repeated PET (up to once per year). Additional patient information (addended to that described above) including severity of COVID-19, persistence of symptoms, and interval between infection and time of scan will be documented. We will determine the SUVmax values for tissues including brain, eye, nose, throat, parotid, thyroid, esophagus, lung, myocardium, liver, gallbladder, pancreas, spleen, renal cortex, renal medulla, duodenum, jejunum, ileum, breast, ovary, testes, penis, bone marrow, and adrenal glands; and compare uptake values for patients across the cohorts. We anticipate elevated PET-derived signals in lung, liver, spleen, endocrine, and reproductive tissues, and decreased renal uptake. We anticipate all COVID-19 patients to follow these trends (compared to healthy subjects data) and that long-COVID-19 subjects (who had not returned to baseline health) will display the largest deviation from healthy controls.

What is claimed is:

1. An imaging agent comprising an ACE2 binding peptide, a radiolabel, and a chelator.

2. The imaging agent of claim 1, wherein the ACE2 binding peptide comprises a linear DX600 peptide or a cyclized DX600 peptide.

3. The imaging agent of claim 2, wherein the ACE2 binding peptide comprises an amino acid sequence, Gly-Asp-Tyr-Ser-His-Cys-Ser-Pro-Leu-Arg-Tyr-Tyr-Pro-Trp-Trp-Lys-Cys-Thr-Tyr-Pro-Asp-Pro-Glu-Gly-Gly-Gly (SEQ ID NO: 1).

4. The imaging agent of claim 3, wherein ACE2 binding peptide further comprises one or more chemical modifications that confer resistance to proteolysis.

5. The imaging agent of claim 4, wherein the amino acid sequence further comprises one or more conservative substitutions.

6. The imaging agent of claim 4, wherein the radiolabel comprises any one of ²H (D or deuterium), ³H (T or tritium), ¹¹C, ¹³C, ¹⁴C, ⁶⁴Cu, ⁶⁷Cu, ¹⁷⁷Lu, ¹³N, ¹⁵N, ¹⁵O, ¹⁷O, ¹⁸O, ¹⁸F, ⁸⁹Sr, ³⁵S, ¹⁵³Sm, ³⁶Cl, ⁸²Br, ⁷⁵Br, ⁷⁶Br, ⁷⁷Br, ¹²³I, ¹²⁴I, ¹²⁵I, ¹³¹I, ¹¹¹In, ⁶⁷Ga, ⁶⁸Ga, ¹⁷⁷Lu, ¹⁸⁶Re, ¹⁸⁸Re, ²⁰¹Tl, ^{99m}Tc, ⁹⁰Y, ⁸⁹Zr, ²²⁵Ac, ²¹³Bi, ²¹²Pb, ²²⁷Th, ¹⁵⁰Gd, ¹⁵²Gd, ¹⁵³Gd, ¹⁵⁴Gd, ¹⁵⁵Gd, ¹⁵⁶Gd, ¹⁵⁷Gd, ¹⁵⁸Gd, ¹⁶⁰Gd, and ²²³Ra.

7. The imaging agent of claim 6, wherein the radiolabel comprises any one of ⁶⁴Cu, ⁶⁸Ga, and ¹⁸F.

8. The imaging agent of claim 7, wherein the chelator comprises any one of NHS-MAG3, MAG3, DTPA, 3p-C-NE3TA, 3p-C-NOTA, 3p-C-DE4TA, ATSM, NOTA (1,4,7-triazacyclononane-N,N′,N″-triacetic acid), NODAGA (1,4,7-triazacyclononane,1-glutaric acid-4,7-acetic acid), DOTA (1,4,7,10-tetraazacyclododecane-1,4,7,10-tetraacetic acid), DOTA-NHS, pSCN-Bn-DOTA, pNH₂—Bn-DOTA, TETA (1,4,8,11-tetraazacyclotetradecane-1,4,8,11-tetraacetic acid, TETA-octreotide (OC), DOTAGA (1,4,7,10-tetraazacyclododecane,1-(glutaric acid)-4,7,10-triacetic acid)), hexaazamacrobicyclic cage-type ligands (e.g., Sarcophogine chelators), cross-bridged tetraamine ligands (e.g., CB-TE2A (4,11-bis(carboxymethyl)-1,4,8,11-tetraazabicyclo[6.6.2]hexadecane)), 6-Hydrazinopridine-3-carboxylic acid (Hynic), NHS-Hynic, and 2,2′,2″-(10-(2-((2-(2,5-dioxo-2,5-dihydro-1H-pyrrol-1-yl)ethyl)amino)-2-oxoethyl)-1,4,7,10-tetraazacyclododecane-1,4,7-triyl)triacetic acid (Maleimido-mono-amide-DOTA).

9. The imaging agent of claim 8, wherein the chelator is NODAGA (1,4,7-triazacyclononane,1-glutaric acid-4,7-acetic acid), NOTA (1,4,7-triazacyclononane-N,N′,N″-triacetic acid) or DOTA (1,4,7,10-tetraazacyclododecane-1,4,7,10-tetraacetic acid).

10. The imaging agent of claim 9, wherein the chelator is conjugated to the ACE2 binding peptide and the chelator is radiolabeled.

11. The imaging agent of claim 10, wherein the imaging agent is a PET or SPECT imaging agent selected from ⁶⁴Cu-NODAGA-DX600, ⁶⁴Cu-NOTA-DX600, or ⁶⁸Ga-DOTA-DX600.

12. The imaging agent of claim 11, wherein the imaging agent detects an ACE2 expression profile.

13. A method of detecting an ACE2 expression profile in a subject, the method comprising:
administering to the subject an imaging agent comprising an ACE2 binding peptide, a radiolabel, and a chelator; and
detecting the imaging agent.

14. The method of claim 13, wherein detecting the imaging agent comprises imaging the subject using a detection method comprising at least one of: positron emission tomography (PET) imaging, single-photon emission computed tomography (SPECT) imaging, mass spectrometry, gamma imaging, magnetic resonance imaging (MRI), magnetic resonance spectroscopy imaging, fluorescence spectroscopy imaging, CT imaging, ultrasound imaging, photoacoustic imaging, and X-ray imaging.

15. A method of selecting a treatment for an ACE2 associated disease for a subject, the method comprising:
administering to a subject an imaging agent comprising an ACE2 binding peptide, a radiolabel, and a chelator;
detecting the imaging agent; and
selecting a treatment based on the detected imaging agent.

16. The method of claim 15, wherein selecting a treatment based on the detected imaging agent further comprises selecting a treatment for the subject if the ACE2 expression rate is elevated above a threshold rate.

17. The method of claim 15, wherein selecting a treatment based on the detected imaging agent further comprises selecting a treatment for the subject if ACE2 expression is detected within a subject's lungs.

18. The method of claim 15, wherein the ACE2 associated disease is a disease, disorder, or condition selected from the group consisting of: a cardiovascular disorder, a renal pathophysiology, diabetes, a lung disorder, an ACE2-associated coronavirus infection, and a cancer.

19. The method of claim 18, wherein the ACE2-associated coronavirus infection includes an infection by at least one of a severe acute respiratory syndrome (SARS)-CoV virus, a human CoV-NL63 virus, and a severe acute respiratory syndrome coronavirus clade 2 (SARS-CoV-2) virus.

20. A pharmaceutical composition comprising an imaging agent comprising an ACE2 binding peptide, a radiolabel, and a chelator.
